(19) United States
(12) Patent Application Publication
(10) Pub. No.: US 2022/0267812 A1
(43) Pub. Date: Aug. 25, 2022

(54) MULTIPLEXED ENGINEERED CELLS AND SYSTEMS FOR BIOFUEL PRODUCTION

(71) Applicant: Inscripta, Inc., Boulder, CO (US)

(72) Inventors: Andrew Garst, Boulder, CO (US); Daniel Held, Boulder, CO (US); Eric Abbate, Boulder, CO (US); Katherine Noon, Boulder, CO (US)

(21) Appl. No.: 17/676,720

(22) Filed: Feb. 21, 2022

Related U.S. Application Data
(60) Provisional application No. 63/151,740, filed on Feb. 21, 2021.
(60) Provisional application No. 63/163,293, filed on Mar. 19, 2021.

(51) Int. Cl. C12P 7/10; C12N 15/10; C12N 1/20; C12N 15/70; C12N 9/22; C12N 15/11; C12N 15/52; C10L 1/02

(52) U.S. Cl. CPC C12P 7/10; C12N 15/1058; C12N 1/20; C12N 15/70; C12N 9/22; C12N 15/11; C12N 15/52; C10L 1/02; C12N 2310/20; C12N 2800/80; C12N 2800/101; C10L 2290/26

(57) ABSTRACT

The present disclosure provides multiplexed engineered cells, automated multi-module instruments and methods of producing biofuel producing cells for enhanced production of biofuels. This platform empowers users to design genetic variant libraries of insertions, swaps, and/or deletions, that can be intentionally or randomly positioned across the entire genome to generate engineered cell populations with improved properties for several common biofuel applications including, but not limited to, improved tolerance to biomass inhibitors, increased thermo-tolerance, increased ethanol production and/or tolerance, expanded carbon utilization abilities, and increased utilization of heterologous proteins or pathways.

### RELATED CASES

This application claims priority to U.S. Ser. No. 63/151,740, filed 21 Feb. 2021, and U.S. Ser. No. 63/163,293, filed 19 Mar. 2021, both of which are incorporated herein in their entirety.

### FIELD OF THE INVENTION

The present disclosure relates to methods and compositions to improve the generation of biofuels in a cell population when using nucleic-acid guided editing, as well as automated multi-module instruments for performing these methods and using these compositions.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

One of the major challenges faced in commercial production of biofuels from engineered microorganisms is the management of various toxic compounds generated during the thermo-chemical treatment steps of biomass. These toxic compounds can interfere with, for example, the production of the biofuels by the microorganisms. As the global demand for renewable energy solutions grows, the field of biofuels research has continued to seek solutions to meet these demands. Identifying new systems for producing biofuels requires a platform and methods for using the same that enable multiplex genetic engineering of biofuels-relevant strains by libraries of gene insertions, gene swaps, and/or gene deletions, which can be engineered across the entire genome or at specific locations of interest. The present disclosure addresses this need.

### SUMMARY OF ILLUSTRATIVE EMBODIMENTS

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, and automated multi-module cell processing instruments for the editing of cells for bioproduction of biofuels from populations of genetically engineered cells. The disclosure includes methods of using nucleic-acid guided editing in cell populations, e.g., bacterial and fungal cells, for creation of cell populations for enhanced bioproduction of biofuels. In some aspects, the disclosure provides processes for generating engineered cells for the improved bioconversion of lignocellulosic biomass to desired biofuels. In some aspects, the disclosures comprise methods to identify cells edited with a library of vectors that solve technical challenges in lignocellulosic material processing. In particular, the disclosure provides methods for identifying edited cells that have increased tolerance to acetic acid, a major inhibitor derived from lignocellulosic biomass. The present disclosure provides methods, including automated methods, for increasing the efficient production of naturally occurring biofuels from organic materials such as lignocellulose.

In specific aspects, the disclosure provides cells produced by the disclosed methods, where the cells are designed to efficiently produce a biofuel, such as ethanol (traditionally derived from corn), biodiesel (traditionally derived from vegetable oils and liquid animal fats), green diesel (traditionally derived from algae and other plant sources), and biogas (methane traditionally derived from animal manure and other digested organic material).

In some aspects, the cells edited using the methods of the disclosure are microbial cells which serve as mini “factories” to produce the desired biofuel.

In other aspects, the cells are edited with genome-wide multiplex CRISPR systems for producing site saturation, heterologous expression of biofuel pathway genes, and/or inhibitory endogenous gene knock-outs that would be advantageous for biofuels research, e.g., yeast cells that are edited to have a tolerance to products formed during pre-treatment and fermentation of lignocellulosic substrates used in ethanol bioproduction. In other aspects, the cells are edited to create organisms that express one or more components of a molecular pathway required for the manufacture of the biofuel by the cell, e.g., a genetically engineered strain of Saccharomyces cerevisiae having a gene which confers resistance to the by-products of ethanol production by the yeast.

The cells that can be edited using the methods of the disclosure include any prokaryotic, archaeal or eukaryotic cell. For example, prokaryotic cells for use with the present illustrative embodiments can be gram-positive bacterial cells or gram-negative bacterial cells, e.g., E. coli cells. Eukaryotic cells for use with the automated multi-module cell editing instruments of the illustrative embodiments include any plant cells and any animal cells, e.g. fungal cells (including yeast), insect cells, amphibian cells, and the like.

In some aspects, the cells are engineered to contain a heterologous pathway for the bioproduction of a biofuel. In specific aspects, the heterologous pathway is introduced into a model organism including but not limited to as E. coli, S. cerevisiae, Streptomyces, Pseudomonas, B. subtilis, or Pichia pastoris. In specific aspects, heterologous genes encoding for substances with known biofuel activity are engineered into common research microbial species such as E. coli or S. cerevisiae.

In some aspects, the microbes edited using the methods of the disclosure include, bacterial cells. In some aspects, the microbes edited using the methods of the disclosure include fungal cells.

In some aspects, the genes of organisms with an ability to produce biofuels (or mutations found in homologous genes from these organisms) can be introduced as heterologous sequences into cells of another genera, species or strain. Accordingly, in some aspects, genes from one organism are heterologously introduced into another organism for genetic manipulation and optimization. The genes could either be integrated into the new host genome or carried on a plasmid.

In some aspects, cellular pathways with known ability to produce compounds with biofuel activity (e.g., Saccharomyces cerevisiae strains) can be used as the “starting” point for selection of genes that are genetically engineered into the genome of a microorganism for improved production of a biofuel. Saccharomyces cerevisiae for instance, is a robust fermentative yeast but has limitations as a potential cellulosic ethanol production (CBP) host, such as low heterologous protein secretion titers. Described herein is a process for engineering strains, including S. cerevisiae strains, for superior biofuel secretion activity and other industrially relevant characteristics needed during the process of lignocellulosic ethanol production.

Following production of the Mattel the biofuel can preferably be isolated from the modified cells or host organism producing the biofuel.

In certain embodiments, automated methods are used for nuclease-directed genome editing of one or more target genomic regions in multiple cells for enhancement of the ability of these cells to produce biofuels, the methods being performed in automated multi-module cell editing instruments. These methods can be used to generate libraries of living cells of interest with desired genomic changes or to use libraries of genes in multiplex gene editing screening panels that identify endogenous genes associated with a sensitivity or a tolerance to the biofuel. The automated methods carried out using the automated multi-module cell editing instruments described herein can be used with a variety of nuclease-directed genome editing techniques, and can be used with or without use of one or more selectable markers.

The present disclosure thus provides, in selected embodiments, modules, instruments, and systems for automated multi-module cell editing for enhancement of a cell's ability to produce, for example by fermentation, one or more biofuels. Automated systems for cell editing that may be used for can be found, e.g., in U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; U.S. Pat. No. 10,738,663, issued 29 Sep.2020; and U.S. Pat. No. 10,894,958, issued 19 Jan. 2021 all of which are herein incorporated by reference in their entirety.

In specific aspects, there is provided an automated multi-module cell editing instrument for enhancement of a cell's ability to produce one or more biofuels, including via nuclease-directed genome editing. Other specific embodiments of the automated multi-module cell editing instruments of the disclosure are designed for recursive genome editing, e.g., sequentially introducing multiple edits into genomes inside one or more cells of a cell population through two or more editing operations within the instruments.

In specific aspects the disclosure provides a method for generating engineered cells for the production of a biofuel comprising: providing a population of cells; processing the population of cells using an instrument for multiplexed nuclease-directed genome editing using introduced nucleic acids and a nucleic acid-directed nuclease to create cells, wherein the introduced nucleic acids comprise nucleic acids derived from a target library; incubating the processed cells to facilitate nucleic acid editing in the cell; and selecting for edited cells displaying an improved production of the biofuel. In preferred cases, the instrument for multiplexed nuclease-directed genome editing allows a user to design genetic variant libraries of insertions, swaps, and/or deletions that can be intentionally or randomly positioned across the entire genome or at specific locations of interest in the population of cells. Such methods allow for editing of the population of cells with the target library—and subsequent screening of one or more edited strain variants for several phenotypes beneficial to common biofuels applications including, but not limited to, improving tolerance to biomass inhibitors, increasing thermo-tolerance, increasing ethanol production and/or tolerance, expanding carbon utilization, and increasing utilization of heterologous proteins or pathways. In one specific embodiment, the selecting selects for a cell that has improved resistance to an inhibitor generated in the production of the biofuel, such as selection of an edited cell that has an improved tolerance to the acetic acid generated in the production of ethanol from yeast. In other specific embodiments, the selecting selects for a cell that has improved thermo-tolerance for the production of the biofuel. In some cases, the selecting selects for a cell that has tolerance to a toxicity of the biofuel itself. In particular cases, the selecting selects for a cell that has a tolerance to a fermentation of a lignocellulosic substrate. In preferred cases the nuclease is an RNA-directed nuclease, such as MAD7, Cas 9, Cas12/Cpf1. The target library can be a targeted library for generation of gene knock-outs, gene knock-ins, gene swaps, or partial gene deletions in the population of cells. Ultimately, the targeted library is utilized along with the automated multiplex editing system of the disclosure for the discovery of genetically engineered strains that have one or more improved properties in the manufacture of a biofuel, including improved tolerance to biomass inhibitors, increasing thermo-chemical tolerance to compounds used in the pretreatment of the biomass, increasing ethanol production and/or tolerance, expanding carbon utilization, improved secretion of the biofuel from cells, and increasing utilization of heterologous proteins or pathways.

In preferred instances, the disclosure provides a biofuel producing cell, wherein the cell is engineered via CRISPR mediated gene editing to encode a non-endogenous biofuel pathway polynucleotide for translation into a functional polypeptide within the biofuel producing cell. In some instances, the biofuel producing cell may lack an endogenous pathway to produce the biofuel. In some cases, two or more, three or more, or four or more non-endogenous biofuel pathway polynucleotides are engineered into the cell by the multiplex gene editing.

These aspects and other features and advantages of the invention are described below in more detail.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for all purposes, including but not limited to describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “biofuel” as used herein refers to a renewable energy source. Traditionally biofuels are derived from a wide variety of materials, such as crops, wood, manure, and some garbage. Collectively, these sources are known as biomass.

The term “lignocellulose” as used herein refers to organic dry matter (biomass), so called lignocellulosic biomass. It is an abundantly available raw material on Earth which has been used for the production of biofuels, mainly bio-ethanol. It is generally composed of carbohydrate polymers (cellulose, hemicellulose), and an aromatic polymer (lignin).

The term “target library” as used herein refers to a gene editing library compatible with the automated systems of the disclosure that is either generated by random mutagenesis sample sequence space or targeted mutagenesis. For instance, random mutagenesis site saturation target libraries of the disclosure can generate gene editing substitutes for each of the 20 possible amino acids (or some subset of them) at a single position, one-by-one, for each gene or for a plurality of genes of a target genome (e.g., Saccharomyces cerevisiae). Targeted mutagenesis site saturation target libraries of the disclosure can generate gene editing substitutes for each of the 20 possible amino acids (or some subset of them) at a single position, one-by-one, for select groups of genes, such as the genes responsible for glucose- and xylose-specific consumption rates of Saccharomyces cerevisiae or another target genome.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

The term “heterologous” refers to the relationship between two or more nucleic acids or protein sequences from different sources, or the relationship between a protein (or nucleic acid) and a host cell from different sources. For example, if the combination of a nucleic acid and a host cell is usually not naturally occurring, the nucleic acid is heterologous to the host cell. A particular sequence is “heterologous” to the cell or organism into which it is inserted.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “intermediate compound” and “intermediates” refer to a product of a synthesis pathway that is not the terminal product, but which is useful for the production of the final intended product. The term “naturally occurring” when used in reference to a toxin refers to a chemical compound or substance produced by a living organism. In the broadest sense, natural products include any substance produced by life, including substrates, enzymes, cofactors, and terminal products (e.g. final intended biofuel) and pathway intermediates of terminal products. The term also encompasses complex extracts and isolated compounds derived from those extracts. In the broadest sense, a chemical or product that is “naturally occurring” includes any substance or combination of substances produced by life. In addition, the term is intended to encompass a substance that forms the structural basis for commercial herbicides, such as an intermediary product.

The term “nickase” as used herein refers to a nuclease that cuts one strand of a double-stranded DNA at a specific recognition nucleotide sequence.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “selectable marker” refers to a gene introduced into a cell, which confers a trait suitable for artificial selection. General use selectable markers are well-known to those of ordinary skill in the art. For examples, selectable markers can use means that deplete a cell population to enrich for editing, and include ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed. In addition, selectable markers include physical markers that confer a phenotype that can be utilized for physical or computations cell enrichment, e.g., optical selectable markers such as fluorescent proteins (e.g., green fluorescent protein, blue fluorescent protein) and cell surface handles.

The term “specifically binds” as used herein includes an interaction between two molecules, e.g., an engineered peptide antigen and a binding target, with a binding affinity represented by a dissociation constant of about 10⁻⁷ M, about 10⁻⁸ M, about 10⁻⁹ M, about 10⁻¹⁰ M, about 10⁻¹¹M, about 10⁻¹²M, about 10⁻¹³M, about 10⁻¹⁴M or about 10⁻¹⁵ M.

The terms “target genomic DNA sequence”, “cellular target sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In the present disclosure, the term “editing vector” includes a coding sequence for a nuclease, a gRNA sequence to be transcribed, and a donor DNA sequence. In other embodiments, however, two vectors—an engine vector comprising the coding sequence for a nuclease, and an editing vector, comprising the gRNA sequence to be transcribed and the donor DNA sequence—may be used.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A-1C depict an automated multi-module instrument and components thereof with which to practice the recursive editing methods as taught herein.

FIG. 2A depicts one embodiment of a rotating growth vial for use with the cell growth module described herein. FIG. 2B illustrates a perspective view of one embodiment of a rotating growth vial in a cell growth module. FIG. 2C depicts a cut-away view of the cell growth module from FIG. 2B. FIG. 2D illustrates the cell growth module of FIG. 2B coupled to LED, detector, and temperature regulating components.

FIG. 3A is a model of tangential flow filtration used in the TFF device presented herein. FIG. 3B depicts a top view of a lower member of one embodiment of an exemplary TFF device. FIG. 3C depicts a top view of upper and lower members and a membrane of an exemplary TFF device. FIG. 3D depicts a bottom view of upper and lower members and a membrane of an exemplary TFF device. FIGS. 3E-3I depict various views of yet another embodiment of a TFF module having fluidically coupled reservoirs.

FIGS. 4A shows a flow-through electroporation device exemplary (here, there are six such devices co-joined). FIG. 4B is a top view of one embodiment of an exemplary flow-through electroporation device. FIG. 4C depicts a top view of a cross section of the electroporation device of FIG. 4C. FIG. 4D is a side view cross section of a lower portion of the electroporation devices of FIGS. 4C and 4D.

FIGS. 5A and 5B depict the structure and components of one embodiment of a reagent cartridge.

FIG. 6 is a simplified block diagram of an embodiment of an exemplary automated multi-module cell processing instrument.

FIG. 7 is a schematic depicting mechanical, thermo-chemical, enzymatic, and microbial steps that are part of traditional microbial fermentation of sugars from sugarcane and corn starch to ethanol.

### BRIEF DESCRIPTION

This disclosure is directed to the editing of organisms for enhanced biosynthesis of naturally occurring biofuels using engineered microbes. Lignocellulosic biomass, for example, is an abundant and renewable resource naturally occurring in plants that is mainly composed of polysaccharides (cellulose and hemicelluloses) and an aromatic polymer (lignin). This starting material has a high potential as an alternative to fossil resources to produce second-generation biofuels and biosourced chemicals and materials without compromising global food security. However, the bioconversion of lignocellulosic biomass to desired products must be improved to reach economic viability. One of the main technical hurdles to lignocellulosic biomass production is the presence of inhibitors in biomass hydrolysates, which hampers the bioconversion efficiency by biorefinery microbial platforms such as—e.g., Saccharomyces cerevisiae—in terms of both production yields and rates. The technical challenges can be divided into structural factors (cellulose specific surface area, cellulose crystallinity, degree of polymerization, pore size and volume) and chemical factors (composition and content in lignin, hemicelluloses, acetyl groups). In particular, acetic acid, a major inhibitor derived from lignocellulosic biomass, severely restrains the performance of engineered xylose-utilizing S. cerevisiae strains, resulting in decreased cell growth, xylose utilization rate, and product yield. The present disclosure provides methods, including automated methods, for increasing the efficient production of naturally occurring biofuels from organic materials such as lignocellulose.

Commercially, there are a number of advantages in using naturally occurring lignocellulosic biomass in biofuel production, especially as a sustainable alternative to conventional fossil-derived fuels. First, it is a highly abundant material. Second, it potentially offers a sustainable supply of biofuel. Third, it does not compete with the food sources of the fermenting microorganism for energy supply (e.g., it does not compete with xylose-utilization by S. cerevisiae strains). Fourth, the composition of the biomass is well understood; lignocellulosic biomass primarily consists of three polymers, cellulose, hemicellulose, and lignin, along with small amounts of substituents such as acetyl groups. However, lignocellulose conversion to ethanol by microbial fermentation generates toxic or inhibitory by-products that are detrimental to the yeast performance, since it must face high concentrations of toxic chemicals and harmful process conditions, Thus, yeast cells can be exposed to inhibitory concentrations of toxic chemicals and low pH resulting from thermo-chemical pretreatment of lignocellulose. Furthermore, saccharification and fermentation of sugar polymers expose fermenting yeast to high temperatures, elevated osmolarity, and high concentrations of the target biofuel—ethanol. Thus, generation of novel microorganisms capable of resisting conditions of lignocellulose ethanol production processes whereas maintaining high metabolic activity are desirable. Although microbial strains with improved characteristics for the generation of the biofuel ethanol in particular have been isolated from natural habitats where they have been evolving these traits (Ballesteros et al., 1991; Edgardo et al., 2008; Field et al., 2015), the exiting strains do not have the phenotypes required for large scale biofuel synthesis.

Provided herein are methods for generating engineered cells for the production of a biofuel, preferably yeast cells, where the cells are tolerant to one or more toxic or inhibitory by-products of biofuel generation, comprising: providing a population of cells; processing the population of cells using an instrument for multiplexed nuclease-directed genome editing using introduced nucleic acids and a nucleic acid-directed nuclease to create cells, wherein the introduced nucleic acids comprise a library of vectors for either targeted knock-out of genes inhibitory to biofuel generation, incubating the processed cells to facilitate nucleic acid editing in the cell; and selecting for edited cells displaying an improved production of the biofuel.

In some aspects the disclosure provides an automated platform for: a) generation of cell populations with numerous parallel mutations; b) high-throughput screening; and c) selection of cell population with desirable characteristics for biofuel processing. Such characteristics include, for example, better performance in the inhibitory conditions found in lignocellulosic ethanol production processes, and better regulation of molecular responses following sensing, signal transduction, signal integration, and execution of cellular functions in response to environmental stresses. Results from systems biology. The disclosure provides methods for evaluating the performance of genetically edited cells from multiple different stresses found in a typical biofuel production process.

In some aspects the disclosure provides biofuel producing cells engineered via CRISPR mediated gene editing to encode a non-endogenous biofuel pathway polynucleotide for translation into a functional polypeptide within the biofuel producing cell. In some aspects, these cells are engineered to have heterologous expression of coding and non-coding genes that are beneficial for the production of biofuels, such as cellulase genes, genes that provide a high secretory phenotype for the biofuel as compared to a wild type cell, or genes that provide a high fermentative capacity in the engineered cell. In select instances, the cell is engineered to have a tolerance to products formed during pre-treatment and fermentation of lignocellulosic substrates. In specific instances, the biofuel producing cell lacks an endogenous pathway to produce the biofuel. The cell can be a microbial cell, a bacterial cell, a fungal cell, or a plant cell. In some cases, the non-endogenous biofuel pathway polynucleotide encodes a gene in an alcohol synthesis pathway, such as genes beneficial for diesel or ethanol synthesis pathway. Similarly, the non-endogenous biofuel pathway polynucleotide may encode a gene in a biogas synthesis pathway. As described elsewhere herein, the cell may be edited with CRISPR mediated gene multiplex gene editing. Preferably, two or more, three or more, or four or more non-endogenous biofuel pathway polynucleotides are engineered into the cell by such editing.

### Nuclease-Directed Genome Engineering

The compositions and methods described herein are employed to perform nuclease-directed genome editing to introduce desired edits to a population of microbial cells. In some embodiments, a single edit is introduced in a single round of editing. In some embodiments, multiple edits are introduced in a single round of editing using simultaneous editing, e.g., the introduction of two or more edits on a single vector. In some embodiments, recursive cell editing is performed where edits are introduced in successive rounds of editing.

A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence (either a cellular target sequence or a curing target sequence). By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. For additional information regarding “CREATE” editing cassettes, see U.S. Pat. Nos. 9,982,278; 10,266,849; 10,240,167; 10,351,877; 10,364,442; 10,435,715; 10,465,207; 10,669,559; 10,711,284; 10,713,180; and 16/938,739, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to the microbial cell, or in vitro. A target sequence can be a sequence encoding a gene product (e.g., a protein) or a non-coding sequence (e.g., a regulatory polynucleotide, an intron, a PAM, a control sequence, etc.).

The guide nucleic acid may be and preferably is part of an editing cassette that encodes the donor nucleic acid that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the donor nucleic acid in, e.g., an editing cassette. In other cases, the donor nucleic acid in, e.g., an editing cassette can be inserted or assembled into a vector backbone first, followed by insertion of the sequence coding for the guide nucleic acid. Preferably, the sequence encoding the guide nucleic acid and the donor nucleic acid are located together in a rationally designed editing cassette and are simultaneously inserted or assembled via gap repair into a linear plasmid or vector backbone to create an editing vector.

The target sequence is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In certain embodiments, the genome editing of a cellular target sequence both introduces a desired DNA change to a cellular target sequence, e.g., the genomic DNA of a cell, and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular microbial cell types, such as stem cells. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease is encoded by a DNA sequence on a vector and optionally is under the control of an inducible promoter. In some embodiments, the promoter may be separate from but the same as the promoter controlling transcription of the guide nucleic acid; that is, a separate promoter drives the transcription of the nuclease and guide nucleic acid sequences but the two promoters may be the same type of promoter. Alternatively, the promoter controlling expression of the nuclease may be different from the promoter controlling transcription of the guide nucleic acid; that is, e.g., the nuclease may be under the control of, e.g., the pTEF promoter, and the guide nucleic acid may be under the control of the, e.g., pCYC1 promoter.

Another component of the nucleic acid-guided nuclease system is the donor nucleic acid comprising homology to the cellular target sequence. The donor nucleic acid is on the same vector and even in the same editing cassette as the guide nucleic acid and preferably is (but not necessarily is) under the control of the same promoter as the editing gRNA (that is, a single promoter driving the transcription of both the editing gRNA and the donor nucleic acid). The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Ser. No. 63/073,287, filed 1 Sep. 2020. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. In many embodiments, the donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence.

As described in relation to the gRNA, the donor nucleic acid can be provided as part of a rationally-designed editing cassette, which is inserted into an editing plasmid backbone where the editing plasmid backbone may comprise a promoter to drive transcription of the editing gRNA and the donor DNA when the editing cassette is inserted into the editing plasmid backbone. Moreover, there may be more than one, e.g., two, three, four, or more editing gRNA/donor nucleic acid rationally-designed editing cassettes inserted into an editing vector; alternatively, a single rationally-designed editing cassette may comprise two to several editing gRNA/donor DNA pairs, where each editing gRNA is under the control of separate different promoters, separate like promoters, or where all gRNAs/donor nucleic acid pairs are under the control of a single promoter. In some embodiments the promoter driving transcription of the editing gRNA and the donor nucleic acid (or driving more than one editing gRNA/donor nucleic acid pair) is optionally an inducible promoter.

In addition to the donor nucleic acid, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library editing gRNAs and of donor nucleic acids representing, e.g., gene-wide or genome-wide libraries of editing gRNAs and donor nucleic acids. The library of editing cassettes is cloned into vector backbones where, e.g., each different donor nucleic acid is associated with a different barcode. Also, in preferred embodiments, an editing vector or plasmid encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

Cells with a stably integrated genomic copy of the GFP gene can enable phenotypic detection of genomic edits of different classes (NHEJ, HDR, no edit) by flow cytometry, fluorescent cell imaging, or genotypic detection by sequencing of the genome-integrated GFP gene. Lack of editing, or perfect repair of cut events in the GFP gene result in cells that remain GFP-positive. Cut events that are repaired by the Non-Homologous End-Joining (NHEJ) pathway often result in nucleotide insertion or deletion events (Indel). These Indel edits often result in frame-shift mutations in the coding sequence that cause loss of GFP gene expression and fluorescence. Cut events that are repaired by the Homology-Directed Repair (HDR) pathway, using the GFP to BFP HDR donor as a repair template result in conversion of the cell fluorescence profile from that of GFP to that of BFP.

### Editing Cassette

The editing cassette used was a plasmid that mediates expression of a gRNA that targets the nuclease to a specific DNA sequence. The editing cassette plasmid can also have or lack a DNA sequence (HDR donor) to provide a template for targeted insertions, deletions, or nucleotide swaps proximal to the nuclease-targeted cut site. In one example, the editing cassette plasmid expresses a gRNA targeting a stably integrated genomic copy of the GFP gene and provides an HDR donor that mediates nucleotide swaps which convert the amino acid coding sequence of GFP to that of BFP.

An RNA-guided nuclease (e.g., Cas9, Cpf1, MAD7) can be delivered to the cell by means of a nuclease-encoding expression plasmid, nuclease-encoding mRNA, recombinant nuclease protein, or by generation of a nuclease-expressing stable cell line. In this specific example, the MAD7 nuclease was delivered by means of a nuclease-encoding expression plasmid.

Automated Cell Editing Instruments and Modules to Perform Nucleic Acid-Guided Nuclease Editing

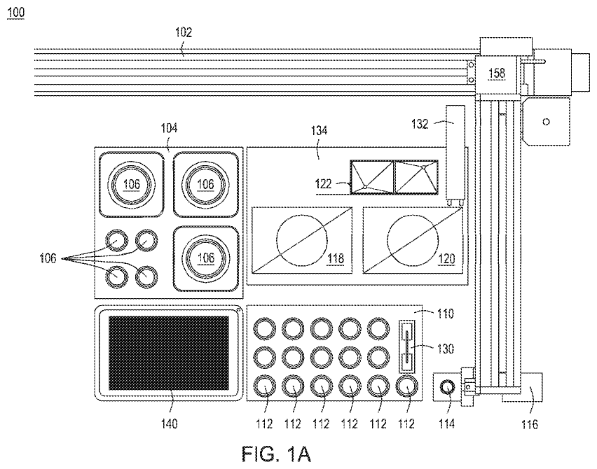

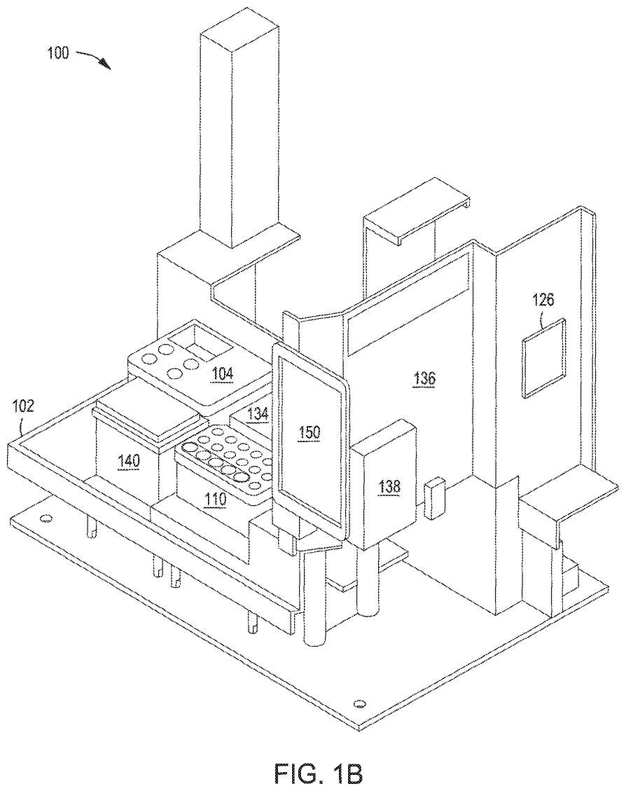

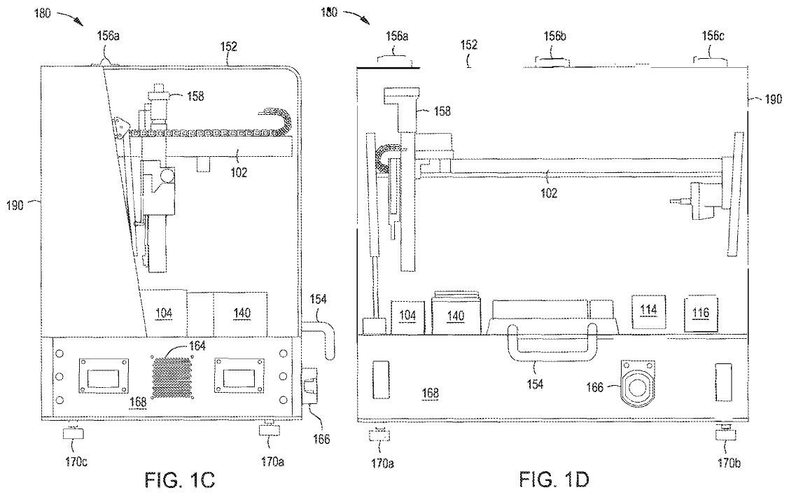

FIG. 1A depicts an exemplary automated multi-module cell processing instrument 100 to, e.g., perform one of the exemplary workflows described herein. The instrument 100, for example, may be and preferably is designed as a desktop instrument for use within a laboratory environment. The instrument 100 may incorporate a mixture of reusable and disposable elements for performing various staged processes in conducting automated genome cleavage and/or editing in cells. Illustrated is a gantry 102, providing an automated mechanical motion system (actuator) (not shown) that supplies XYZ axis motion control to, e.g., an automated liquid handling system 158 including, e.g., an air displacement pipette as well as modules of the automated multi-module cell processing instrument 100. In some automated multi-module cell processing instruments, the air displacement pipettor 132 is moved by gantry 102 and the various modules and reagent cartridges remain stationary; however, in other embodiments, the liquid handling system may stay stationary while the various modules are moved. Also included in the automated multi-module cell processing instrument 100 is reagent cartridge 110 comprising reservoirs 112 and transformation module 130, as well as a wash cartridge 104 comprising reservoirs 106. The wash cartridge 104 may be configured to accommodate large tubes, for example, wash solutions, or solutions that are used often throughout an iterative process. In one example, wash cartridge 104 may be configured to remain in place when two or more reagent cartridges 110 are sequentially used and replaced. Although reagent cartridge 110 and wash cartridge 104 are shown in FIG. 1A as separate cartridges, the contents of wash cartridge 104 may be incorporated into reagent cartridge 110. Note in this embodiment transformation module 130 is contained within reagent cartridge 110; however, in alternative embodiments transformation module 130 is contained within its own module or may be part of another module, such as a growth module.

The wash and reagent cartridges 104 and 110 in some implementations, are disposable kits provided for use in the automated multi-module cell editing instrument 100. For example, a user may open and position each of the reagent cartridge 110 and the wash cartridge 104 within a chassis of the automated multi-module cell editing instrument prior to activating cell processing.

Also illustrated is the robotic handling system 158 including the gantry 102 and air displacement pipettor 132. In some examples, the robotic handling system 158 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 132.

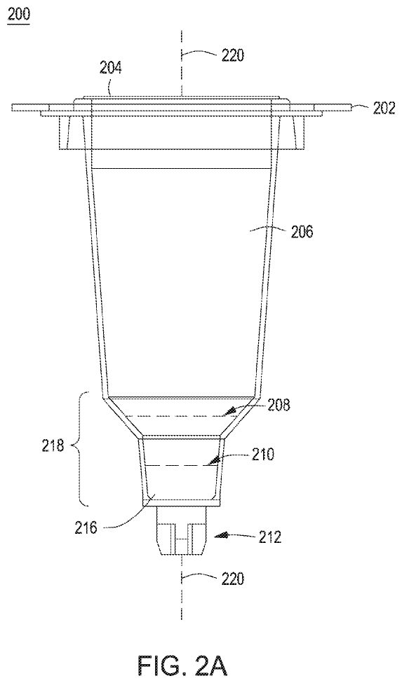

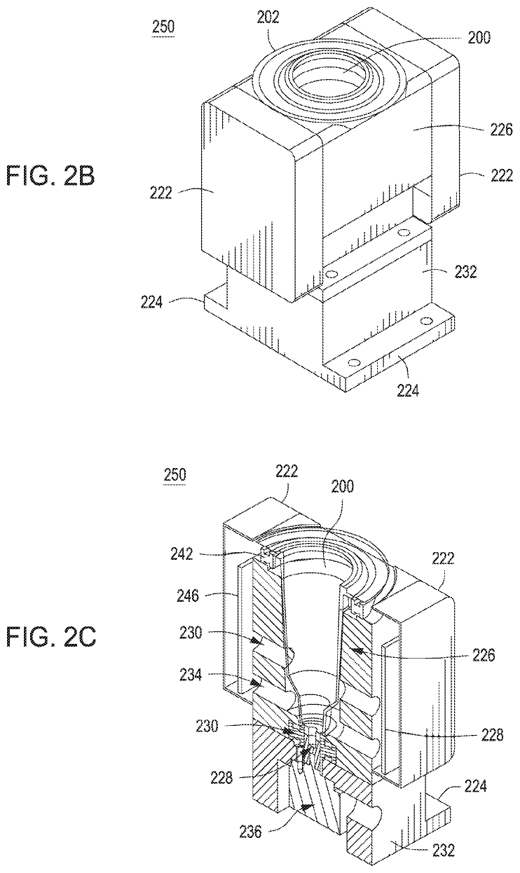

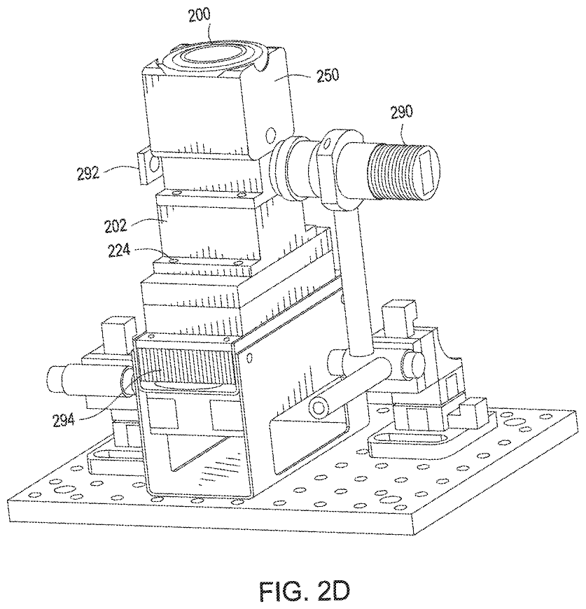

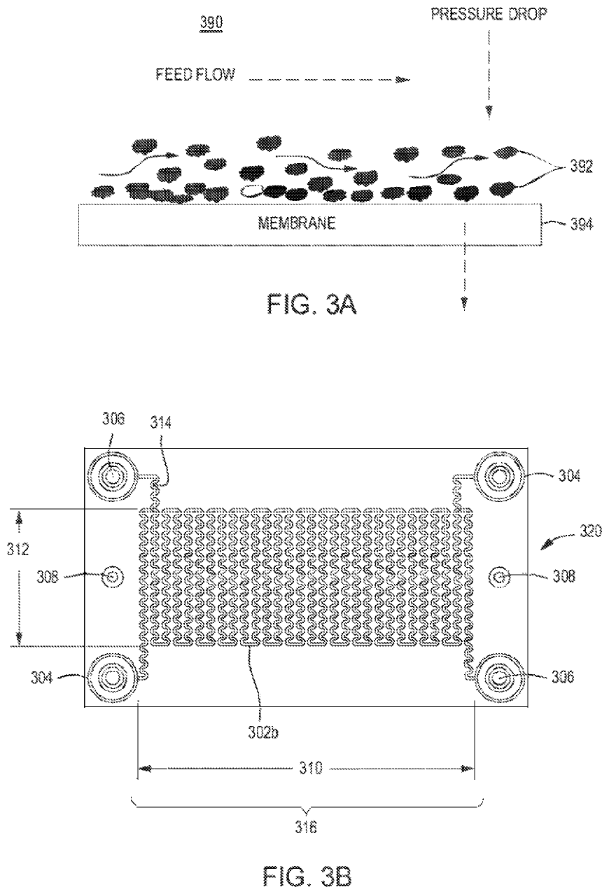

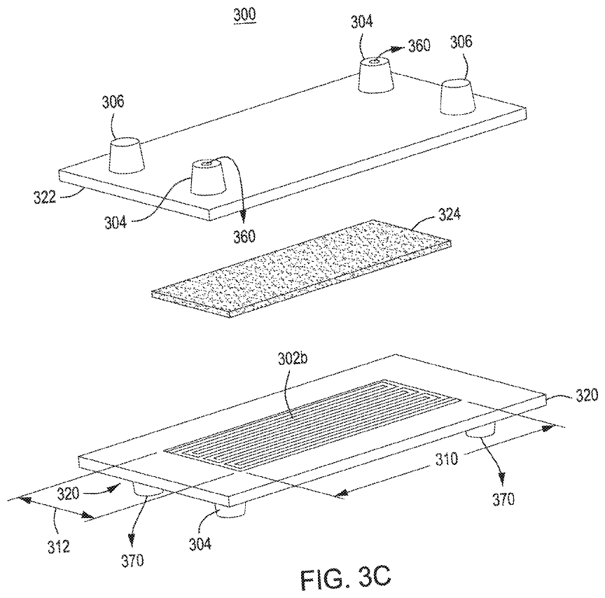

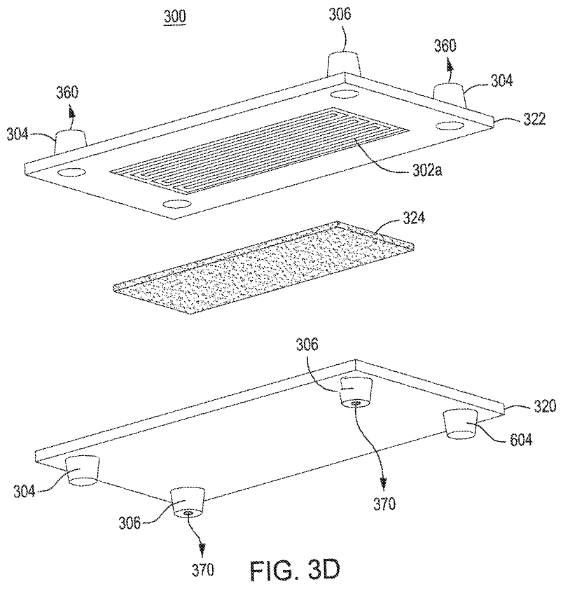

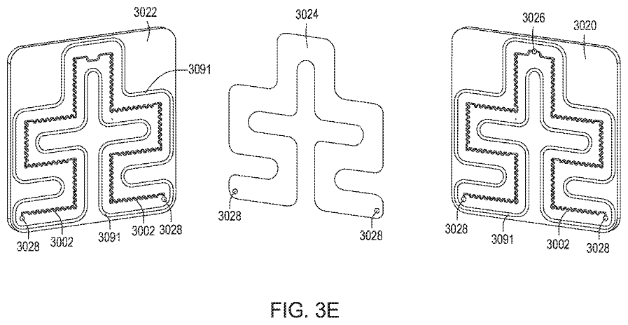

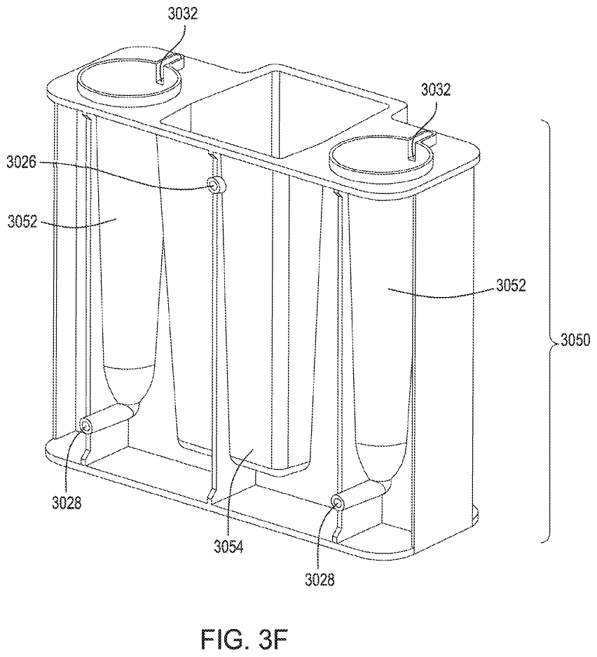

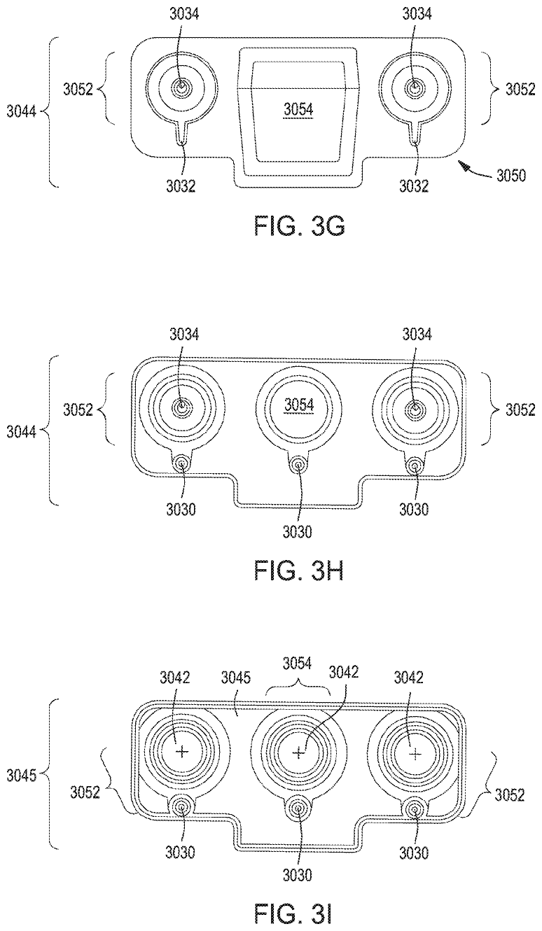

Components of the cartridges 104, 110, in some implementations, are marked with machine-readable indicia (not shown), such as bar codes, for recognition by the robotic handling system 158. For example, the robotic handling system 158 may scan containers within each of the cartridges 104, 110 to confirm contents. In other implementations, machine-readable indicia may be marked upon each cartridge 104, 110, and the processing system 126 (shown in FIG. 1D) of the automated multi-module cell editing instrument 100 may identify a stored materials map based upon the machine-readable indicia. The exemplary automated multi-module cell processing instrument 100 of FIG. 1A further comprises a cell growth module 134. (Note, all modules recited briefly here are described in detail below.) In the embodiment illustrated in FIG. 1A, the cell growth module 134 comprises two cell growth vials 118, 120 (described in greater detail below in relation to FIGS. 2A-2D) as well as a cell concentration module 122 (described in detail in relation to FIGS. 3A-3F). In alternative embodiments, the cell concentration module 122 may be separate from cell growth module 134, e.g., in a separate, dedicated module. Also illustrated as part of the automated multi-module cell processing instrument 100 of FIG. 1A is an optional enrichment module 140, served by, e.g., robotic handing system 158 and air displacement pipettor 132. Also seen are an optional nucleic acid assembly/desalting module 114 comprising a reaction chamber or tube receptacle (not shown) and a magnet 116 to allow for purification of nucleic acids using, e.g., magnetic solid phase reversible immobilization (SPRI) beads (Applied Biological Materials Inc., Richmond, BC). The cell growth module, cell concentration module, transformation module, enrichment module, and reagent cartridge are described in greater detail below.

FIG. 1B is a plan view of the front of the exemplary multi-module cell processing instrument 100 depicted in FIG. 1A. Cartridge-based source materials (such as in reagent cartridge 110), for example, may be positioned in designated areas on a deck 102 of the instrument 100 for access by a robotic handling instrument (not shown in this figure). As illustrated in FIG. 1B, the deck 102 may include a protection sink such that contaminants spilling, dripping, or overflowing from any of the modules of the instrument 100 are contained within a lip of the protection sink. In addition to reagent cartridge 110, also seen in FIG. 1B is wash cartridge 104, optional enrichment module 140, and a portion of growth module 134. Also seen in this view is touch screen display 150, transformation module controls 138, electronics rack 136, and processing system 126.

FIGS. 1C through 1D illustrate multi-module cell processing instruments 180 comprising chassis 190 for use in desktop versions the cell editing instrument 180. For example, the chassis 190 may have a width of about 24-48 inches, a height of about 24-48 inches and a depth of about 24-48 inches. Chassis 190 may be and preferably is designed to hold multiple modules and disposable supplies used in automated cell processing. Further, chassis 190 may mount a robotic handling system 158 for moving materials between modules.

As illustrated, the chassis 190 includes a cover having a handle 154 and hinges 156a-156c for lifting the cover and accessing the interior of the chassis 190. A cooling grate 164 allows for air flow via an internal fan (not shown). Further, the chassis 190 is lifted by adjustable feet 170 (feet 170a-c are shown). The feet 170a-170c, for example, may provide additional air flow beneath the chassis 190. A control button 166, in some embodiments, allows for single-button automated start and/or stop of cell processing within the chassis 190.

Inside the chassis 190, in some implementations, a robotic handling system 158 is disposed along a gantry 102 above materials cartridges 104 and 110. Control circuitry, liquid handling tubes, air pump controls, valves, thermal units (e.g., heating and cooling units) and other control mechanisms, in some embodiments, are disposed below a deck of the chassis 190, in a control box region 168. Also seen in FIG. 1D is enrichment module 140 and nucleic acid assembly module 114 comprising a magnet 116.

Although not illustrated, in some embodiments a display screen may be positioned on the front face of the chassis 190, for example covering a portion of the cover (e.g., see FIG. 1B). The display screen may provide information to the user regarding the processing status of the automated multi-module cell editing instrument. In another example, the display screen may accept inputs from the user for conducting the cell processing.

### The Rotating Cell Growth Module

FIG. 2A shows one embodiment of a rotating growth vial 200 for use with the cell growth device described herein configured to grow all cell types including cells lines and primary cells. The rotating growth vial is an optically-transparent container having an open end 204 for receiving liquid media and cells, a central vial region 206 that defines the primary container for growing cells, a tapered-to-constricted region 218 defining at least one light path 210, a closed end 216, and a drive engagement mechanism 212. The rotating growth vial has a central longitudinal axis 220 around which the vial rotates, and the light path 210 is generally perpendicular to the longitudinal axis of the vial. The first light path 210 is positioned in the lower constricted portion of the tapered-to-constricted region 218. Optionally, some embodiments of the rotating growth vial 200 have a second light path 208 in the tapered region of the tapered-to-constricted region 218. Both light paths in this embodiment are positioned in a region of the rotating growth vial that is constantly filled with the cell culture (cells+growth media) and is not affected by the rotational speed of the growth vial. The first light path 210 is shorter than the second light path 208 allowing for sensitive measurement of OD values when the OD values of the cell culture in the vial are at a high level (e.g., later in the cell growth process), whereas the second light path 208 allows for sensitive measurement of OD values when the OD values of the cell culture in the vial are at a lower level (e.g., earlier in the cell growth process). Also shown is lip 202, which allows the rotating growth vial to be seated in a growth module (not shown) and further allows for easy handling for the user.

In some configurations of the rotating growth vial, the rotating growth vial has two or more “paddles” or interior features disposed within the rotating growth vial, extending from the inner wall of the rotating growth vial toward the center of the central vial region. In some aspects, the width of the paddles or features varies with the size or volume of the rotating growth vial, and may range from 1/20 to just over ⅓ the diameter of the rotating growth vial, or from 1/15 to ¼ the diameter of the rotating growth vial, or from 1/10 to ⅕ the diameter of the rotating growth vial. In some aspects, the length of the paddles varies with the size or volume of the rotating growth vial, and may range from ⅘ to ¼ the length of the main body of the rotating growth vial, or from ¾ to ⅓ the length of the main body of the rotating growth vial, or from ½ to ⅓ the length of the main body of the rotating growth vial. In other aspects, there may be concentric rows of raised features disposed on the inner surface of the main body of the rotating growth vial arranged horizontally or vertically; and in other aspects, there may be a spiral configuration of raised features disposed on the inner surface of the main body of the rotating growth vial. In alternative aspects, the concentric rows of raised features or spiral configuration may be disposed upon a post or center structure of the rotating growth vial. Though described above as having two paddles, the rotating growth vial may comprise 3, 4, 5, 6 or more paddles, and up to 20 paddles. The number of paddles will depend upon, e.g., the size or volume of the rotating growth vial. The paddles may be arranged symmetrically as single paddles extending from the inner wall of the vial into the interior of the vial, or the paddles may be symmetrically arranged in groups of 2, 3, 4 or more paddles in a group (for example, a pair of paddles opposite another pair of paddles) extending from the inner wall of the vial into the interior of the vial. In another embodiment, the paddles may extend from the middle of the rotating growth vial out toward the wall of the rotating growth vial, from, e.g., a post or other support structure in the interior of the rotating growth vial.

The drive engagement mechanism 212 engages with a motor (not shown) to rotate the vial. In some embodiments, the motor drives the drive engagement mechanism 212 such that the rotating growth vial is rotated in one direction only, and in other embodiments, the rotating growth vial is rotated in a first direction for a first amount of time or periodicity, rotated in a second direction (i.e., the opposite direction) for a second amount of time or periodicity, and this process may be repeated so that the rotating growth vial (and the cell culture contents) are subjected to an oscillating motion. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial may be oscillated at a first periodicity (e.g., every 60 seconds), and then a later stage of cell growth the rotating growth vial may be oscillated at a second periodicity (e.g., every one second) different from the first periodicity.

The rotating growth vial 200 may be reusable or, preferably, the rotating growth vial is consumable. In some embodiments, the rotating growth vial is consumable and is presented to the user pre-filled with growth medium, where the vial is hermetically sealed at the open end 204 with a foil seal. A medium-filled rotating growth vial packaged in such a manner may be part of a kit for use with a stand-alone cell growth device or with a cell growth module that is part of an automated multi-module cell processing instrument. To introduce cells into the vial, a user need only pipette up a desired volume of cells and use the pipette tip to punch through the foil seal of the vial. Open end 204 may optionally include an extended lip 202 to overlap and engage with the cell growth device (not shown). In automated systems, the rotating growth vial 200 may be tagged with a barcode or other identifying means that can be read by a scanner or camera that is part of the automated system (not shown).

The volume of the rotating growth vial 200 and the volume of the cell culture (including growth medium) may vary greatly, but the volume of the rotating growth vial 200 must be large enough for the cell culture in the growth vial to get proper aeration while the vial is rotating. In practice, the volume of the rotating growth vial 200 may range from 1-250 ml, 2-100 ml, from 5-80 ml, 10-50 ml, or from 12-35 ml. Likewise, the volume of the cell culture (cells+growth media) should be appropriate to allow proper aeration in the rotating growth vial. Thus, the volume of the cell culture should be approximately 10-85% of the volume of the growth vial or from 20-60% of the volume of the growth vial. For example, for a 35 ml growth vial, the volume of the cell culture would be from about 4 ml to about 27 ml, or from 7 ml to about 21 ml.

The rotating growth vial 200 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include glass, polyvinyl chloride, polyethylene, polyamide, polyethylene, polypropylene, polycarbonate, poly(methyl methacrylate (PMMA), polysulfone, polyurethane, and co-polymers of these and other polymers. Specific materials for use with the present disclosure include polypropylene, polycarbonate, and polystyrene. In some embodiments, the rotating growth vial is inexpensively fabricated by, e.g., injection molding or extrusion.

FIGS. 2B-2D show an embodiment of a cell growth module 250 comprising a rotating growth vial 200. FIG. 2B is a perspective view of one embodiment of a cell growth device 250. FIG. 2C depicts a cut-away view of the cell growth device 250 from FIG. 2B. In both figures, the rotating growth vial 200 is seen positioned inside a main housing 226 with the extended lip 202 of the rotating growth vial 200 extending above the main housing 226. Additionally, end housings 222, a lower housing 232, and flanges 224 are indicated in both figures. Flanges 224 are used to attach the cell growth device to heating/cooling means or other structure (not shown). FIG. 2C depicts additional detail. In FIG. 2C, upper bearing 242 and lower bearing 230 are shown positioned in main housing 226. Upper bearing 242 and lower bearing 230 support the vertical load of rotating growth vial 200. Lower housing 232 contains the drive motor 236. The cell growth device of FIG. 2C comprises two light paths: a primary light path 234, and a secondary light path 230. Light path 234 corresponds to light path 210 positioned in the constricted portion of the tapered-to-constricted portion of the rotating growth vial, and light path 230 corresponds to light path 208 in the tapered portion of the tapered-to-constricted portion of the rotating growth vial. Light paths 210 and 208 are not shown in FIG. 2C but may be seen in, e.g., FIG. 2A. In addition to light paths 234 and 230, there is an emission board 228 to illuminate the light path(s), and detector board 246 to detect the light after the light travels through the cell culture liquid in the rotating growth vial.

The motor 236 used to rotate the rotating growth vial 200 in some embodiments is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 206 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 226, end housings 222 and lower housing 232 of the cell growth device 250 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial is envisioned in some embodiments to be reusable but preferably is consumable, the other components of the cell growth device 250 are preferably reusable and can function as a stand-alone benchtop device or, as here, as a module in a multi-module cell processing system.

The processor (not shown) of the cell growth system may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor of the cell growth system may be programmed to use wavelength values for blanks commensurate with the growth media typically used in microbial cell culture. Alternatively, a second spectrophotometer and vessel may be included in the cell growth system, where the second spectrophotometer is used to read a blank at designated intervals.

FIG. 2D illustrates a cell growth device as part of an assembly comprising the cell growth device of FIG. 2B coupled to light source 290, detector 292, and thermal components 294. The rotating growth vial 200 is inserted into the cell growth device. Components of the light source 290 and detector 292 (e.g., such as a photodiode with gain control to cover 5-log) are coupled to the main housing of the cell growth device. The lower housing 232 that houses the motor that rotates the rotating growth vial is illustrated, as is one of the flanges 224 that secures the cell growth device to the assembly. Also illustrated is a Peltier device or thermoelectric cooler 294. In this embodiment, thermal control is accomplished by attachment and electrical integration of the cell growth device 200 to the thermal device 294 via the flange 204 on the base of the lower housing 232. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 200 is controlled to approximately +/−0.5° C.

In certain embodiments, a rear-mounted power entry module contains the safety fuses and the on-off switch, which when switched on powers the internal AC and DC power supplies (not shown) activating the processor. Measurements of optical densities (OD) at programmed time intervals are accomplished using a 600 nm Light Emitting Diode (LED) (not shown) that has been columnated through an optic into the lower constricted portion of the rotating growth vial which contains the cells of interest. The light continues through a collection optic to the detection system which consists of a (digital) gain-controlled silicone photodiode. Generally, optical density is normally shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the cell growth device OD measurement records the overall power transmission, so as the cells grow and become denser in population the OD (the loss of signal) increases. The OD system is pre-calibrated against OD standards with these values stored in an on-board memory accessible by the measurement program.

In use, cells are inoculated (cells can be pipetted, e.g., from an automated liquid handling system or by a user) into pre-filled growth media of a rotating growth vial by piercing though the foil seal. The programmed software of the cell growth device sets the control temperature for growth, typically 30° C., then slowly starts the rotation of the rotating growth vial. The cell/growth media mixture slowly moves vertically up the wall due to centrifugal force allowing the rotating growth vial to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 250 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 on minutes. While the cell growth device has been described in the context of measuring the optical density (OD) of a growing cell culture, it should, however, be understood by a skilled artisan given the teachings of the present specification that other cell growth parameters can be measured in addition to or instead of cell culture OD. For example, spectroscopy using visible, UV, or near infrared (NIR) light allows monitoring the concentration of nutrients and/or wastes in the cell culture. Additionally, spectroscopic measurements may be used to quantify multiple chemical species simultaneously. Nonsymmetric chemical species may be quantified by identification of characteristic absorbance features in the NIR. Conversely, symmetric chemical species can be readily quantified using Raman spectroscopy. Many critical metabolites, such as glucose, glutamine, ammonia, and lactate have distinct spectral features in the IR, such that they may be easily quantified. The amount and frequencies of light absorbed by the sample can be correlated to the type and concentration of chemical species present in the sample. Each of these measurement types provides specific advantages. FT-NIR provides the greatest light penetration depth and can be used for thicker sample. FT-mid-IR (MIR) provides information that is more easily discernible as being specific for certain analytes as these wavelengths are closer to the fundamental IR absorptions. FT-Raman is advantageous when interference due to water is to be minimized. Other spectral properties can be measured via, e.g., dielectric impedence spectroscopy, visible fluorescence, fluorescence polarization, or luminescence. Additionally, the cell growth device may include additional sensors for measuring, e.g., dissolved oxygen, carbon dioxide, pH, conductivity, and the like.

### The Cell Concentration Module

FIGS. 3A-3I depict variations on one embodiment of a cell concentration/buffer exchange cassette and module that utilizes tangential flow filtration and is configured for use with all cell types, including immortalized cell lines, primary cells and/or stem cells. One embodiment of a cell concentration device described herein operates using tangential flow filtration (TFF), also known as crossflow filtration, in which the majority of the feed flows tangentially over the surface of the filter thereby reducing cake (retentate) formation as compared to dead-end filtration, in which the feed flows into the filter. Secondary flows relative to the main feed are also exploited to generate shear forces that prevent filter cake formation and membrane fouling thus maximizing particle recovery, as described below.

The TFF device described herein was designed to take into account two primary design considerations. First, the geometry of the TFF device leads to filtering the cell culture over a large surface area so as to minimize processing time. Second, the design of the TFF device is configured to minimize filter fouling. FIG. 3A is a general model of tangential flow filtration. The TFF device operates using tangential flow filtration, also known as cross-flow filtration. FIG. 3A shows a system 390 with cells flowing over a membrane 394, where the feed flow of the cells 392 in medium or buffer is parallel to the membrane 394. TFF is different from dead-end filtration where both the feed flow and the pressure drop are perpendicular to a membrane or filter.

FIG. 3B depicts a top view of the lower member of one embodiment of a TFF device/module providing tangential flow filtration. As can be seen in the embodiment of the TFF device of FIG. 3C, TFF device 300 comprises a channel structure 316 comprising a flow channel 302b through which a cell culture is flowed. The channel structure 316 comprises a single flow channel 302b that is horizontally bifurcated by a membrane (not shown) through which buffer or medium may flow, but cells cannot. This particular embodiment comprises an undulating serpentine geometry 314 (i.e., the small “wiggles” in the flow channel 302) and a serpentine “zig-zag” pattern where the flow channel 302 crisscrosses the device from one end at the left of the device to the other end at the right of the device. The serpentine pattern allows for filtration over a high surface area relative to the device size and total channel volume, while the undulating contribution creates a secondary inertial flow to enable effective membrane regeneration preventing membrane fouling. Although an undulating geometry and serpentine pattern are exemplified here, other channel configurations may be used as long as the channel can be bifurcated by a membrane, and as long as the channel configuration provides for flow through the TFF module in alternating directions. In addition to the flow channel 302b, portals 304 and 306 as part of the channel structure 316 can be seen, as well as recesses 308. Portals 304 collect cells passing through the channel on one side of a membrane (not shown) (the “retentate”), and portals 306 collect the medium (“filtrate” or “permeate”) passing through the channel on the opposite side of the membrane (not shown). In this embodiment, recesses 308 accommodate screws or other fasteners (not shown) that allow the components of the TFF device to be secured to one another.

The length 310 and width 312 of the channel structure 316 may vary depending on the volume of the cell culture to be grown and the optical density of the cell culture to be concentrated. The length 310 of the channel structure 316 typically is from 1 mm to 300 mm, or from 50 mm to 250 mm, or from 60 mm to 200 mm, or from 70 mm to 150 mm, or from 80 mm to 100 mm. The width of the channel structure 316 typically is from 1 mm to 120 mm, or from 20 mm to 100 mm, or from 30 mm to 80 mm, or from 40 mm to 70 mm, or from 50 mm to 60 mm. The cross-section configuration of the flow channel 102 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 302 is generally round, oval or elliptical, the radius of the channel may be from about 50 μm to 1000 μm in hydraulic radius, or from 5μm to 800 μm in hydraulic radius, or from 200 μm to 700 μm in hydraulic radius, or from 300 μm to 600 μm wide in hydraulic radius, or from about 200 to 500 μm in hydraulic radius.

When looking at the top view of the TFF device/module of FIG. 3B, note that there are two retentate portals 304 and two filtrate portals 306, where there is one of each type portal at both ends (e.g., the narrow edge) of the device 300. In other embodiments, retentate and filtrate portals can on the same surface of the same member (e.g., upper or lower member), or they can be arranged on the side surfaces of the assembly. Unlike other TFF devices that operate continuously, the TFF device/module described herein uses an alternating method for concentrating cells. The overall workflow for cell concentration using the TFF device/module involves flowing a cell culture or cell sample tangentially through the channel structure. The membrane bifurcating the flow channels retains the cells on one side of the membrane and allows unwanted medium or buffer to flow across the membrane into a filtrate side (e.g., lower member 320) of the device. In this process, a fixed volume of cells in medium or buffer is driven through the device until the cell sample is collected into one of the retentate portals 304, and the medium/buffer that has passed through the membrane is collected through one or both of the filtrate portals 306. All types of prokaryotic and eukaryotic cells—both adherent and non-adherent cells—can be grown in the TFF device. Adherent cells may be grown on beads or other cell scaffolds suspended in medium that flow through the TFF device.

In the cell concentration process, passing the cell sample through the TFF device and collecting the cells in one of the retentate portals 304 while collecting the medium in one of the filtrate portals 306 is considered “one pass” of the cell sample. The transfer between retentate reservoirs “flips” the culture. The retentate and filtrate portals collecting the cells and medium, respectively, for a given pass reside on the same end of TFF device/module 300 with fluidic connections arranged so that there are two distinct flow layers for the retentate and filtrate sides, but if the retentate portal 304 resides on the upper member of device/module 300 (that is, the cells are driven through the channel above the membrane and the filtrate (medium) passes to the portion of the channel below the membrane), the filtrate portal 306 will reside on the lower member of device/module 100 and vice versa (that is, if the cell sample is driven through the channel below the membrane, the filtrate (medium) passes to the portion of the channel above the membrane). This configuration can be seen more clearly in FIGS. 3C-3D, where the retentate flows 360 from the retentate portals 304 and the filtrate flows 370 from the filtrate portals 306.

At the conclusion of a “pass” in the growth concentration process, the cell sample is collected by passing through the retentate portal 304 and into the retentate reservoir (not shown). To initiate another “pass”, the cell sample is passed again through the TFF device, this time in a flow direction that is reversed from the first pass. The cell sample is collected by passing through the retentate portal 304 and into retentate reservoir (not shown) on the opposite end of the device/module from the retentate portal 304 that was used to collect cells during the first pass. Likewise, the medium/buffer that passes through the membrane on the second pass is collected through the filtrate portal 306 on the opposite end of the device/module from the filtrate portal 306 that was used to collect the filtrate during the first pass, or through both portals. This alternating process of passing the retentate (the concentrated cell sample) through the device/module is repeated until the cells have been concentrated to a desired volume, and both filtrate portals can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell concentration may (and typically do) take place simultaneously.

FIG. 3C depicts a top view of upper (322) and lower (320) members of an exemplary TFF module. Again, portals 304 and 306 are seen. As noted above, recesses such as the recesses 308 seen in FIG. 3B—provide a means to secure the components (upper member 322, lower member 320, and membrane 324) of the TFF device/membrane to one another during operation via, e.g., screws or other like fasteners. However, in alterative embodiments an adhesive, such as a pressure sensitive adhesive, or ultrasonic welding, or solvent bonding, may be used to couple the upper member 322, lower member 320, and membrane 324 together. Indeed, one of ordinary skill in the art given the guidance of the present disclosure can find yet other configurations for coupling the components of the TFF device, such as e.g., clamps; mated fittings disposed on the upper and lower members; combination of adhesives, welding, solvent bonding, and mated fittings; and other such fasteners and couplings.

Note that there is one retentate portal and one filtrate portal on each “end” (e.g., the narrow edges) of the TFF device/module. The retentate and filtrate portals on the left side of the device/module will collect cells (flow path at 360) and medium (flow path at 370), respectively, for the same pass. Likewise, the retentate and filtrate portals on the right side of the device/module will collect cells (flow path at 360) and medium (flow path at 370), respectively, for the same pass. In this embodiment, the retentate is collected from portals 304 on the top surface of the TFF device, and filtrate is collected from portals 306 on the bottom surface of the device. The cells are maintained in the TFF flow channel above the membrane 324, while the filtrate (medium) flows through membrane 324 and then through portals 306; thus, the top/retentate portals and bottom/filtrate portals configuration is practical. It should be recognized, however, that other configurations of retentate and filtrate portals may be implemented such as positioning both the retentate and filtrate portals on the side (as opposed to the top and bottom surfaces) of the TFF device. In FIG. 3C, the channel structure 302b can be seen on the bottom member 320 of the TFF device 300. However, in other embodiments, retentate and filtrate portals can reside on the same of the TFF device.

Also seen in FIG. 3C is membrane or filter 324. Filters or membranes appropriate for use in the TFF device/module are those that are solvent resistant, are contamination free during filtration, and are able to retain the types and sizes of cells of interest. For example, pore sizes can be as low as 0.2 μm, however for other cell types, the pore sizes can be as high as 5 μm. Indeed, the pore sizes useful in the TFF device/module include filters with sizes from 0.20 μm, 0.21 μm, 0.22 μm, 0.23 μm, 0.24 οm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching. The TFF device shown in FIGS. 3C and 3D do not show a seat in the upper 312 and lower 320 members where the filter 324 can be seated or secured (for example, a seat half the thickness of the filter in each of upper 312 and lower 320 members); however, such a seat is contemplated in some embodiments.

FIG. 3D depicts a bottom view of upper and lower components of the exemplary TFF module shown in FIG. 3C. FIG. 3D depicts a bottom view of upper (322) and lower (320) components of an exemplary TFF module. Again portals 304 and 306 are seen. Note again that there is one retentate portal and one filtrate portal on each end of the device/module. The retentate and filtrate portals on the left side of the device/module will collect cells (flow path at 360) and medium (flow path at 370), respectively, for the same pass. Likewise, the retentate and filtrate portals on the right side of the device/module will collect cells (flow path at 360) and medium (flow path at 370), respectively, for the same pass. In FIG. 3D, the channel structure 302a can be seen on the upper member 322 of the TFF device 300. Thus, looking at FIGS. 3C and 3D, note that there is a channel structure 302 (302a and 302b) in both the upper and lower members, with a membrane 324 between the upper and lower portions of the channel structure. The channel structure 302 of the upper 322 and lower 320 members (302a and 302b, respectively) mate to create the flow channel with the membrane 324 positioned horizontally between the upper and lower members of the flow channel thereby bifurcating the flow channel.

Medium exchange (during cell growth) or buffer exchange (during cell concentration or rendering the cells competent) is performed on the TFF device/module by adding fresh medium to growing cells or a desired buffer to the cells concentrated to a desired volume; for example, after the cells have been concentrated at least 20-fold, 30-fold, 40-fold, 50-fold, 60-fold, 70-fold, 80-fold, 90-fold, 100-fold, 150-fold, 200-fold or more. A desired exchange medium or exchange buffer is added to the cells either by addition to the retentate reservoir or thorough the membrane from the filtrate side and the process of passing the cells through the TFF device 300 is repeated until the cells have been grown to a desired optical density or concentrated to a desired volume in the exchange medium or buffer. This process can be repeated any number of desired times so as to achieve a desired level of exchange of the buffer and a desired volume of cells. The exchange buffer may comprise, e.g., glycerol or sorbitol thereby rendering the cells competent for transformation in addition to decreasing the overall volume of the cell sample.

The TFF device 300 may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 3E depicts an exemplary configuration of an assembled TFF device, where, like the other configurations, the upper member and lower member in combination form a channel structure with a membrane disposed between the upper and lower members; however, in this configuration in addition to the retentate reservoirs, there is in addition an optional buffer or medium reservoir positioned between the retentate reservoirs, and a lower filtrate or permeate reservoir. In the TFF device 3000 configuration shown in FIG. 3E, 3044 is the top or cover of the TFF device 3000, having three ports 3046, where there is a pipette tip 3048 disposed in the right-most port 3046. The top 3044 of the TFF device 3000 is adjacent to and in operation is coupled with a combined reservoir and upper member structure 3050. Combined reservoir and upper member structure 3050 comprises a top surface that is adjacent the top or cover 3044 of the TFF device, a bottom surface which comprises the upper member 3022 of the TFF device, where the upper member 3022 of the TFF device defines the upper portion of the flow channel (not shown) disposed on the bottom surface of the upper member 3022 of the combined reservoir and upper member structure 3050. Additionally, combined reservoir and upper member structure 3050 comprises two retentate reservoirs 3080 and an optional buffer or medium reservoir 3082.

The retentate reservoirs are fluidically coupled to the upper portion of the flow channel, and the buffer or medium reservoir is fluidically coupled to the retentate reservoirs. Also seen in this assembled view of TFF device 3000 is membrane 3024, lower member 3020 which, as described previously comprises on its top surface the lower portion of the tangential flow channel (not shown), where the channel structures of the upper member 3022 and lower member 3020 (neither shown in this view) mate to form a single flow channel. Beneath and adjacent to lower member 3020 is a gasket 3040, which is interposed between lower member 3020 and an optional filtrate (or permeate) reservoir 3042. The filtrate reservoir 3042 is in fluid connection with the lower portion of the flow channel, as a receptacle for the filtrate or permeate that is removed from the cell culture. In operation, top 3044, combined reservoir and upper member structure 3050, membrane 3024, lower member 3020, gasket 3040, and filtrate reservoir 3042 are coupled and secured together to be fluid- and air-tight. The assembled TFF device 3000 typically is from 4 to 25 cm in height, or from 5 to 20 cm in height, or from 7 to 15 cm in height; from 5 to 30 cm in length, or from 8 to 25 cm in length, or from 10 to 20 cm in length; and is from 3 to 15 cm in depth, or from 5 to 10 cm in depth. An exemplary TFF device is 11 cm in height, 12 cm in length, and 8 cm in depth. The retentate reservoirs, buffer or medium reservoir, and tangential flow channel-forming structures may be configured to be cooled to 4° C. for cell maintenance. The dimensions for the serpentine channel recited above, as well as the specifications and materials for the filter and the TFF device apply to the embodiment of the device shown in FIGS. 3E-3I. In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or filtered.

FIG. 3F depicts an exploded perspective view of TFF device 3000. In this configuration, 3044 is the top or cover of the TFF device 3000, having three ports 3046, where there is a pipette tip 3048 disposed in the left-most port 3046. The top 3044 of the TFF device 3000 is, in operation, coupled with a combined reservoir and upper member structure 3050. Combined reservoir and upper member structure 3050 comprises a top surface that, in operation, is adjacent the top or cover 3044 of the TFF device, a bottom surface which comprises the upper member 3022 of the TFF device, where the upper member 3022 of the TFF device defines the upper portion of the tangential flow channel (not shown). Combined reservoir and upper member structure 3050 comprises two retentate reservoirs 3080 and an optional buffer or medium reservoir 3082. The retentate reservoirs are fluidically coupled to the upper portion of the flow channel, and the optional buffer or medium reservoir is fluidically coupled to the retentate reservoirs. Also seen in this exploded view of TFF device 3000 is lower member 3020 which, as described previously comprises on its top surface the lower portion of the tangential flow channel 3002b (seen on the top surface of lower member 3020), where the upper and lower portions of the channel structures of the upper member 3022 and lower member 3020, respectively, when coupled mate to form a single flow channel (the membrane that is interposed between the upper member 3022 and lower member 3020 in operation is not shown). Beneath lower member 3020 is gasket 3040, which in operation is interposed between lower member 3020 and a filtrate (or permeate) reservoir 3042. In operation, top 3044, combined reservoir and upper member structure 3050, membrane (not shown), lower member 3020, gasket 3040, and filtrate reservoir 3042 are coupled and secured together to be fluid- and air-tight. In FIG. 3F, fasteners are shown that can be used to couple the various structures (top 3044, combined reservoir and upper member structure 3050, membrane (not shown), lower member 3020, gasket 3040, and filtrate reservoir 3042) together. However, as an alternative to screws or other like fasteners, the various structures of TFF device 3000 can be coupled using an adhesive, such as a pressure sensitive adhesive; ultrasonic welding; or solvent bonding. Further, a combination of fasteners, adhesives, and/or welding types may be employed to couple the various structures of the TFF device. One of ordinary skill in the art given the guidance of the present disclosure could find yet other configurations for coupling the components of TFF device 3000, such as e.g., clamps, mated fittings, and other such fasteners.

FIG. 3G depicts combined reservoir and upper member structure 3050, comprising two retentate reservoirs 3080 and an optional buffer or medium reservoir 3082, as well as upper member 3020, which is disposed on the bottom of combined reservoir and upper member structure 3050. Upper member 3022 of the TFF device defines the upper portion of the tangential flow channel (not shown) disposed on the bottom surface of the combined reservoir and upper member structure 3050. FIG. 3H is a top-down view of the upper surface of combined reservoir and upper member structure 3050, depicting the top of retentate reservoirs 3080 and buffer or medium reservoir 3082, as well as fluid or vacuum ports 3046. The retentate reservoirs are fluidically coupled to the upper portion of the flow channel, and the buffer or medium reservoir is fluidically coupled to the retentate reservoirs. FIG. 3I is a bottom-up view of the lower surface of combined reservoir and upper member structure 3050, showing the upper member 3020 with the upper portion of the tangential flow channel 3002a disposed on the bottom surface of upper member 3020. The flow channel 3002a disposed on the bottom surface of upper member 3020 in operation is mated to the bottom portion of the tangential flow channel disposed on the top surface of the lower member (not shown in this view, but see FIG. 3F), where the upper and lower portions of the flow channel structure mate to form a single flow channel.

As an alternative to the TFF module described above, a cell concentration module comprising a hollow filter may be employed. Examples of filters suitable for use in the present invention include membrane filters, ceramic filters and metal filters. The filter may be used in any shape; the filter may for example be cylindrical or essentially flat. Preferably, the filter used is a membrane filter, preferably a hollow fiber filter. The term “hollow fiber” is meant a tubular membrane. The internal diameter of the tube is at least 0.1 mm, more preferably at least 0.5 mm, most preferably at least 0.75 mm and preferably the internal diameter of the tube is at most 10 mm, more preferably at most 6 mm, most preferably at most 1 mm. Filter modules comprising hollow fibers are commercially available from various companies, including G.E. Life Sciences (Marlborough, Mass.) and InnovaPrep (Drexel, Mo.). Specific examples of hollow fiber filter systems that can be used, modified or adapted for use in the present methods and systems include, but are not limited to, U.S. Pat. Nos. 9,738,918; 9,593,359; 9,574,977; 9,534,989; 9,446,354;. 9,295,824; 8,956,880; 8,758,623; 8,726,744; 8,677,839; 8,677,840; 8,584,536; 8,584,535; and 8,110,112.

### The Cell Transformation Module

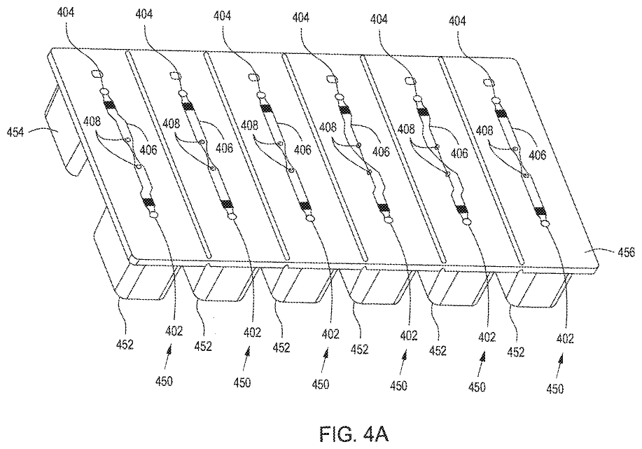

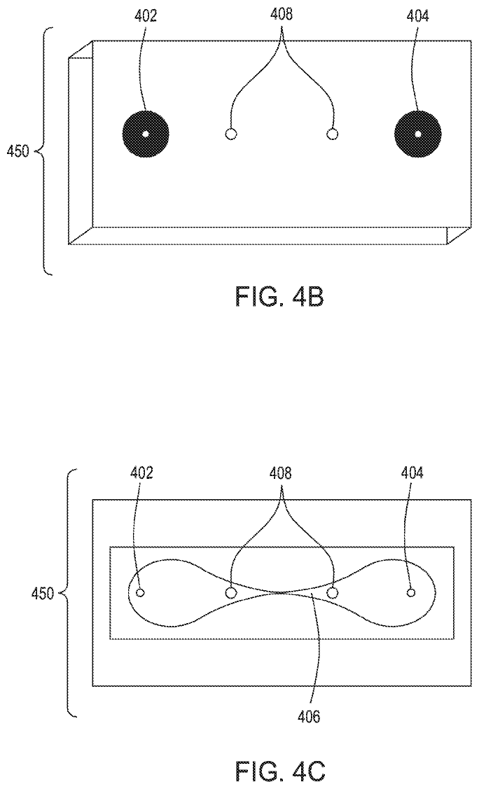

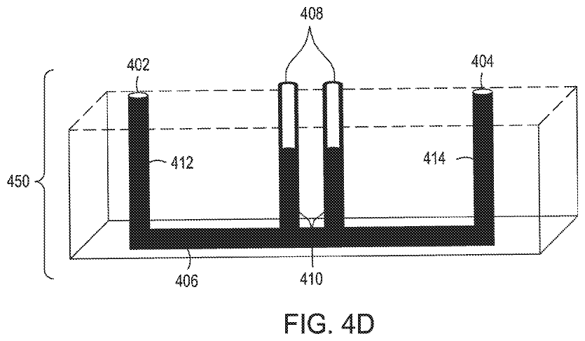

In addition to the modules for cell growth, and cell concentration FIGS. 4A-4E depict variations on one embodiment of a cell transformation module (in this case, a flow through electroporation device) configured to transform microbial cells that may be included in a cell growth/concentration/transformation instrument. FIGS. 4A and 4B are top perspective and bottom perspective views, respectively, of six co-joined flow-through electroporation devices 450. FIG. 4A depicts six flow-through electroporation units 450 arranged on a single substrate 456. Each of the six flow-through electroporation units 450 have wells 452 that define cell sample inlets and wells 454 that define cell sample outlets. FIG. 4B is a bottom perspective view of the six co-joined flow-through electroporation devices of FIG. 4A also depicting six flow-through electroporation units 450 arranged on a single substrate 456. Six inlet wells 452 can be seen, one for each flow-through electroporation unit 450, and one outlet well 454 can be seen (the outlet well of the left-most flow-through electroporation unit 450). Additionally seen in FIG. 4B are an inlet 402, outlet 404, flow channel 406 and two electrodes 408 on either side of a constriction in flow channel 406 in each flow-through electroporation unit 450. Once the six flow-through electroporation units 450 are fabricated, they can be separated from one another (e.g., “snapped apart”) and used one at a time, or alternatively in embodiments two or more flow-through electroporation units 450 can be used in parallel without separation.

The flow-through electroporation devices achieve high efficiency cell electroporation with low toxicity. The flow-through electroporation devices of the disclosure allow for particularly easy integration with robotic liquid handling instrumentation that is typically used in automated systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

Generally speaking, microfluidic electroporation—using cell suspension volumes of less than approximately 10 ml and as low as 1 μl—allows more precise control over a transfection or transformation process and permits flexible integration with other cell processing tools compared to bench-scale electroporation devices. Microfluidic electroporation thus provides unique advantages for, e.g., single cell transformation, processing and analysis; multi-unit electroporation device configurations; and integrated, automatic, multi-module cell processing and analysis.

In specific embodiments of the flow-through electroporation devices of the disclosure the toxicity level of the transformation results in greater than 10% viable cells after electroporation, preferably greater than 15%, 20%, 25%, 30%, 35%, 40%, 45%, 50%, 55%, 60%, 70%, 75%, 80%, 85%, 90%, or even 95% viable cells following transformation, depending on the cell type and the nucleic acids being introduced into the cells.

The flow-through electroporation device described in relation to FIGS. 4A-4E comprises a housing with an electroporation chamber, a first electrode and a second electrode configured to engage with an electric pulse generator, by which electrical contacts engage with the electrodes of the electroporation device. In certain embodiments, the electroporation devices are autoclavable and/or disposable, and may be packaged with reagents in a reagent cartridge. The electroporation device may be configured to electroporate cell sample volumes between 1 μl to 2 ml, 10 μl to 1 ml, 25 μl to 750 μl, or 50 μl to 500 μl.

In one exemplary embodiment, FIG. 4C depicts a top view of a flow-through electroporation device 450 having an inlet 402 for introduction of cells and an exogenous reagent to be electroporated into the cells (“cell sample”) and an outlet 404 for the cell sample following electroporation. Electrodes 408 are introduced through electrode channels (not shown) in the device. FIG. 4D shows a cutaway view from the top of flow-through electroporation device 450, with the inlet 402, outlet 404, and electrodes 408 positioned with respect to a constriction in flow channel 406. In this aspect, the inlet channel, outlet channel and electrode channels all originate from the top planar side of the device; however, the flow-through electroporation architecture depicted in FIGS. 4C-4D is but one architecture useful with the reagent cartridges described herein. Additional electrode architectures are described, e.g., in U.S. Ser. No. 16/147,120, filed 24 Sep. 2018; Ser. No. 16/147,865, filed 30 Sep. 2018; and Ser. No. 16/147,871, filed 30 Sep. 2018.

### The Reagent Cartridge

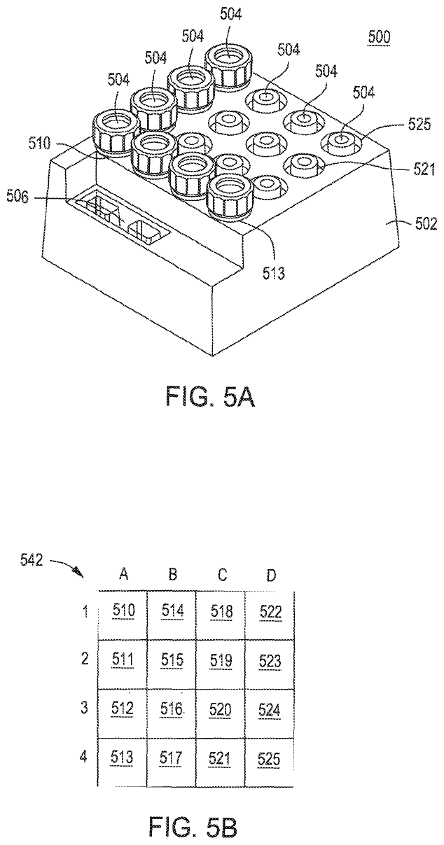

FIG. 5A depicts an exemplary combination reagent cartridge and electroporation device 500 (“cartridge”) that may be used in an automated multi-module cell processing instrument. Cartridge 500 comprises a body 502, and reagent receptacles or reservoirs 504. Additionally, cartridge 500 comprises an electroporation device 506 (an exemplary embodiment of which is described in detail in relation to FIGS. 4A-4D), which is preferably a flow-through electroporation device. Cartridge 500 may be disposable, or may be configured to be reused. Preferably, cartridge 500 is disposable. Cartridge 500 may be made from any suitable material, including stainless steel, aluminum, or plastics including polyvinyl chloride, cyclic olefin copolymer (COC), polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the cartridge is disposable, preferably it is made of plastic. Preferably the material used to fabricate the cartridge is thermally conductive, as in certain embodiments the cartridge 500 contacts a thermal device (not shown) that heats or cools reagents in the reagent receptacles or reservoirs 504. In some embodiments, the thermal device is a Peltier device or thermoelectric cooler. Reagent receptacles or reservoirs 504 may be receptacles into which individual tubes of reagents are inserted as shown in FIG. 5A, receptacles into which one or more multiple co-joined tubes are inserted, or the reagent receptacles may hold the reagents without inserted tubes with the reagents dispensed directly into the receptacles or reservoirs. Additionally, the receptacles in a reagent cartridge may be configured for any combination of tubes, co-joined tubes, and direct-fill of reagents.

In one embodiment, the reagent receptacles or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all receptacles may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. The reagents contained in the reagent cartridge will vary depending on workflow; that is, the reagents will vary depending on the processes to which the cells are subjected in the automated multi-module cell processing instrument.

FIG. 5B depicts an exemplary matrix configuration 140 for the reagents contained in the reagent cartridges of FIG. 5A; where this matrix embodiment is a 4×4 reagent matrix. Through a matrix configuration, a user (or programmed processor) can locate the proper reagent for a given process. That is, reagents such as cell samples, enzymes, buffers, nucleic acid vectors, expression cassettes, reaction components (such as, e.g., MgCl₂, dNTPs, isothermal nucleic acid assembly reagents, Gap Repair reagents, and the like), wash solutions, ethanol, and magnetic beads for nucleic acid purification and isolation, etc. are positioned in the matrix 540 at a known position. For example, reagents are located at positions A1 (510), A2 (511), A3 (512), A4 (513), B1 (514), B2 (515) and so on through, in this embodiment, position D4 (525). FIG. 5A is labeled to show where several reservoirs 504 correspond to matrix 540: See receptacles 510, 513, 521 and 525. Although the reagent cartridge 500 of FIG. 5A and the matrix configuration 540 of FIG. 5B shows a 4×4 matrix, matrices of the reagent cartridge and electroporation devices can be any configuration, such as, e.g., 2×2, 2×3, 2×4, 2×5, 2×6, 3×3, 3×5, 4×6, 6×7, or any other configuration, including asymmetric configurations, or two or more different matrices depending on the reagents needed for the intended workflow. Note in FIG. 4A the matrix configuration is a 5×3+1 matrix.

In preferred embodiments of reagent cartridge 500 shown in FIG. 5A, the reagent cartridge comprises a script (not shown) readable by a processor (not shown) for dispensing the reagents via a liquid handling device (not shown) and controlling the electroporation device contained within reagent cartridge 500. Also, the reagent cartridge 500 as one component in an automated multi-module cell processing instrument may comprise a script specifying two, three, four, five, ten or more processes performed by the automated multi-module cell processing instrument, or even specify all processes performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components of the automated multi-module cell processing instrument may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument such as described in relation to FIGS. 1A-1D. For example, the reagent cartridge may comprise a script to pipette electrocompetent cells from reservoir A2 (511), transfer the cells to the electroporation device 506, pipette a nucleic acid solution comprising an editing vector from reservoir C3 (520), transfer the nucleic acid solution to the electroporation device, initiate the electroporation process for a specified time, then move the porated cells to a reservoir D4 (525) in the reagent cassette or to another module such as the rotating growth vial (118 or 120 of FIG. 1A) in the automated multi-module cell processing instrument in FIG. 1A. In another example, the reagent cartridge may comprise a script to pipette transfer of a nucleic acid solution comprising a vector from reservoir C3 (520), nucleic acid solution comprising editing oligonucleotide cassettes in reservoir C4 (521), and isothermal nucleic acid assembly reaction mix from A1 (510) to the isothermal nucleic acid assembly/desalting reservoir (414 of FIG. 4A). The script may also specify process steps performed by other modules in the automated multi-module cell processing instrument. For example, the script may specify that the isothermal nucleic acid assembly/desalting module be heated to 50° C. for 30 min to generate an assembled isothermal nucleic acid product; and desalting of the assembled isothermal nucleic acid product via magnetic bead-based nucleic acid purification involving a series of pipette transfers and mixing of magnetic beads in reservoir B2 (515), ethanol wash in reservoir B3 (516), and water in reservoir C1 (518) to the isothermal nucleic acid assembly/desalting reservoir (114 of FIG. 1A).

### Use of the Automated Multi-Module Microbial Cell Processing Instrument

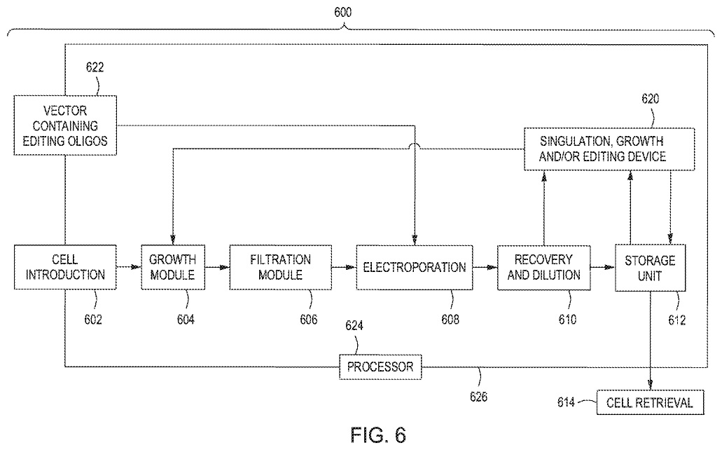

FIG. 6 illustrates an embodiment of a multi-module cell processing instrument. This embodiment depicts an exemplary system that performs recursive gene editing on a microbial cell population. The cell processing instrument 600 may include a housing 626, a reservoir for storing cells to be transformed or transfected 612, and a cell growth module (comprising, e.g., a rotating growth vial) 604. The cells to be transformed are transferred from a reservoir to the cell growth module to be cultured until the cells hit a target OD. Once the cells hit the target OD, the growth module may cool or freeze the cells for later processing or transfer the cells to a cell concentration module 606 where the cells are subjected to buffer exchange and rendered electrocompetent, and the volume of the cells may be reduced substantially. Once the cells have been concentrated to an appropriate volume, the cells are transferred to electroporation device 608. In addition to the reservoir for storing cells 612, the multi-module cell processing instrument includes a reservoir for storing the vector pre-assembled with editing oligonucleotide cassettes 622. The pre-assembled nucleic acid vectors are transferred to the electroporation device 608, which already contains the cell culture grown to a target OD. In the electroporation device 608, the nucleic acids are electroporated into the cells. Following electroporation, the cells are transferred into an optional recovery module 610, where the cells recover briefly post-transformation.

After recovery, the cells may be transferred to a storage module 612, where the cells can be stored at, e.g., 4° C. for later processing, or the cells may be diluted and transferred to an incubation and growth module 620. In some aspects, the cells are transferred from the storage module to a retrieval reservoir 614.

In the incubation and growth module 620, the cells are arrayed such that there is an average of one cell per microwell. The arrayed cells may be in selection medium to select for cells that have been transformed or transfected with the editing vector(s). Once singulated, the cells grow through 2-50 doublings and establish colonies. Editing is then initiated and allowed to proceed, colonies of cells are allowed to grow to terminal size (e.g., normalization of the colonies) in the microwells and then are treated to conditions that cure the editing vector from this round. Once cured, the cells can be flushed out of the microwells and pooled, then transferred to the storage (or recovery) unit 612 or can be transferred back to the growth module 604 for another round of editing. In between pooling and transfer to a growth module, there typically is one or more additional steps, such as cell recovery, medium exchange (rendering the cells electrocompetent), cell concentration (typically concurrently with medium exchange by, e.g., filtration. Note the selection/singulation/growth/incubation/editing/normalization and curing modules may be the same module, where all processes are performed in, e.g., a solid wall device, or selection and/or dilution may take place in a separate vessel before the cells are transferred to the growth/editing module. In some aspects, the cells are singulated or partitioned in smaller cell groups (e.g., 2-600 cells) for growth and/or editing, as described in, e.g., U.S. Pat. Nos. 10,253,316 and 10,532,324.

In other aspects, the cells may be pooled after normalization, transferred to a separate vessel, and cured in the separate vessel. As an alternative to singulation in, e.g., a solid wall device, the transformed cells may be grown in—and editing can proceed in—bulk liquid as described above in U.S. Ser. No. 68/795,739, filed 23 Jan. 2019. Once the putatively-edited cells are pooled, they may be subjected to another round of editing, beginning with growth, cell concentration and treatment to render electrocompetent, and transformation by yet another donor nucleic acid in another editing cassette via the electroporation module 608.

In electroporation device 608, the microbial cells selected from the first round of editing are transformed by a second set of editing oligos (or other type of oligos) and the cycle is repeated until the cells have been transformed and edited by a desired number of, e.g., editing cassettes. The multi-module cell processing instrument exemplified in FIG. 6 is controlled by a processor 624 configured to operate the instrument based on user input or is controlled by one or more scripts including at least one script associated with the reagent cartridge. The processor 624 may control the timing, duration, and temperature of various processes, the dispensing of reagents, and other operations of the various modules of the instrument 600. For example, a script or the processor may control the dispensing of cells, reagents, vectors, and editing oligonucleotides; which editing oligonucleotides are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 6, then the resulting edited culture may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells.

Curing can be accomplished by, e.g., cleaving the vector(s) using a curing plasmid thereby rendering the editing and/or engine vector (or single, combined engine/editing vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine vector.

### Library Design and Nuclease-Directed Genome Editing

One of the major challenges for economic conversion of biofuels is the ability of host cells to cope with the process. For example, a major challenge in the conversion of lignocellulose to fuel ethanol is to generate robust S. cerevisiae strains able to cope with inhibitory conditions while keeping proper catalytic functions for raw material conversion to ethanol. Major inhibitory conditions found in the unit operations required for the conversion processes include the accumulation of toxic chemicals generated during lignocellulose pretreatment and sugar fermentation, the high temperature that accompanied simultaneous saccharification and fermentation, and the very high osmolality and elevated solids loadings at the beginning of the fermentation. Since unification of these unit operations is desirable to reduce production costs and energy utilization, it can be expected that yeast cells will be simultaneously exposed to most of these inhibitory conditions. It should also be noted that the biofuel molecule(s) themselves being produced by the engineered cells can be inhibitory to cell growth. Thus, in some aspects, the disclosure provides methods for generating microbial strain that are better able to cope with biofuel molecule itself.

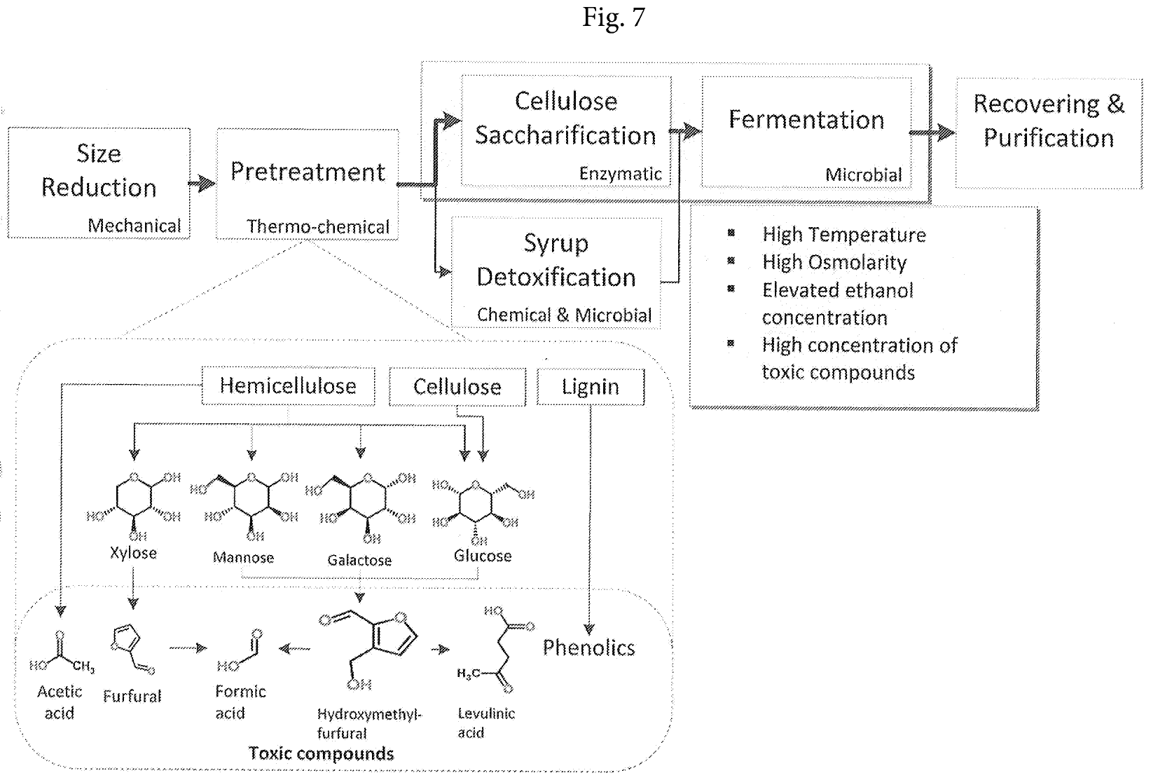

Existing studies attempting to establish metabolic engineering strategies for increasing host tolerance considered the route and regulation of molecular responses following sensing, signal transduction, signal integration, and execution of cellular functions in response to environmental stresses. Results from systems biology and -omics analyses, as well as from traditional data mining, point out the relevance of the cross-regulation between the routes of yeast responses according to the different types of stress, but do not offer a mechanism that is suitable for the large scale screening of complex multiplexed libraries that may be involved in multiple distinct pathways. See, e.g., FIG. 7 for a schematic of steps in the biomass production edits that can be selected for, including edits that improve tolerance to biomass inhibitors, increase thermo-tolerance, increase ethanol production and/or tolerance, expand carbon utilization, and increasing utilization of heterologous proteins or pathways.

For bacterial expression, the engine plasmid can comprise a coding sequence for the MAD7 nuclease under the control of the inducible pL promoter, the λ Red operon recombineering system under the control of the inducible pBAD promoter (inducible by the addition of arabinose in the cell growth medium), the c1857 gene under the control of a constitutive promoter, as well as a selection marker and an origin of replication. As described above, the λ Red recombineering system repairs the double-stranded breaks resulting from the cut by the MAD7 nuclease. The c1857 gene at 30° C. actively represses the pL promoter (which drives the expression of the MAD7 nuclease and the editing or CREATE cassette on the editing cassette such as the exemplary editing vector illustrated on, see U.S. Pat. Nos. 9,982,278; 10,266,849; 10,240,167; 10,351,877; 10,364,442; 10,435,715; 10,465,207; 10,669,559; 10,711,284; 10,731,180 and U.S. Ser. No. 16/550,092; and 16/938,739, all of which are incorporated by reference herein. However, at 42° C., the c1857 repressor gene unfolds or degrades, and in this state the c1857 repressor protein can no longer repress the pL promoter leading to active transcription of the coding sequence for the MAD7 nuclease and the editing (e.g., CREATE) cassette. The CREATE exemplary cassette depicts an exemplary editing plasmid comprising the editing (e.g, CREATE) cassette (crRNA, spacer and HA) driven by a pL promoter, a selection marker, and an origin of replication.

The 200,000 nucleic acid mutations or edits described herein were generated using MAD7, along with a gRNA and donor DNA. A nucleic acid-guided nuclease such as MAD7 is complexed with an appropriate synthetic guide nucleic acid in a cell and can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the methods to generate the 200,000 member library, the guide nucleic acids were provided as a sequence to be expressed from a plasmid or vector comprising both the guide sequence and the scaffold sequence as a single transcript under the control of an inducible promoter. The guide nucleic acids are engineered to target a desired target sequence by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a proto spacer adjacent motif (PAM) sequence adjacent to the target sequence. The target sequences for the genome-wide mutagenesis here encompassed 200,000 unique edits throughout the E. coli genome.

The guide nucleic acid may be generating the variants reported herein were part of an editing cassette that also encoded the donor nucleic acid. The target sequences are associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence.

In certain embodiments, the genome editing of a cellular target sequence both introduces the desired DNA change to the cellular target sequence and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease is encoded by a DNA sequence on a vector (e.g., the engine vector) and be under the control of an inducible promoter. In some embodiments—such as in the methods described herein—the inducible promoter may be separate from but the same as the inducible promoter controlling transcription of the guide nucleic acid; that is, a separate inducible promoter drives the transcription of the nuclease and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

Another component of the nucleic acid-guided nuclease system is the donor nucleic acid comprising homology to the cellular target sequence. In some embodiments, the donor nucleic acid is on the same polynucleotide (e.g., editing vector or editing cassette) as the guide nucleic acid. The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. In many embodiments, the donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence. Various types of edits were introduced herein, including site-directed mutagenesis, saturation mutagenesis, promoter swaps and ladders, knock-in and knock-out edits, SNP or short tandem repeat swaps, and start/stop codon exchanges.

In addition to the donor nucleic acid, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library gRNAs and of donor nucleic acids representing, e.g., gene-wide or genome-wide libraries of gRNAs and donor nucleic acids. The library of editing cassettes is cloned into vector backbones where, e.g., each different donor nucleic acid is associated with a different barcode.

### Enrichment for Selection of Edited Cells

In specific aspects, the disclosure provides methods for improving nuclease-directed editing of cells using enrichment means to identify cells that have received the editing components needed to perform the intended editing operation.

In some aspects, the enrichment handle and method can be based on a positive versus negative signal of the surrogate. In other aspects, the enrichment method can be based on a threshold level of a surrogate, e.g., a high level of an enrichment handle versus a low or absent level of an enrichment handle.

In some aspects, the disclosure provides methods for improving nuclease-directed editing rates by enriching for microbial cells that have received an HDR donor, e.g., identifying cells that are more likely to have received the editing.

In specific aspects of the invention, the HDR is improved using fusion proteins that retain certain characteristics of RNA-directed nucleases (e.g., CRISPR nucleases) and also utilize other enzymatic activities, e.g., replication inhibition, reverse transcriptase activity, transcription enhancement activity, and the like. These nuclease fusion proteins can be used in nuclease-directed editing using the disclosed methods, with or without the enrichment methods as disclosed herein.

In certain aspects, the protocols utilize both the RNA-directed nuclease fusion proteins and a selection. Use of fusion proteins and enrichment for these editing methods may involve a single enrichment method for HDR donor, the guide nucleic acid, and the nuclease, or two or more separate enrichment events for one or more of these elements. The HDR donor and guide nucleic acid may be introduced separately or covalently linked, as disclosed in, e.g., U.S. Pat. No. 9,982,278.

In specific aspects, the cells receiving the HDR donor can be enriched using an initial enrichment step, e.g., using an antibiotic selection or fluorescent detection, following by an enrichment step using an enrichment of the cells receiving and expressing the co-introduced cell surface antigen.

### Exemplary Biofuels and Biofuel Conversion

Biofuels—Ethanol

Ethanol (CH₃CH₂OH) is a renewable fuel that can be made from various plant materials, collectively known as “biomass.” Ethanol is an alcohol used as a blending agent, generally mixed with gasoline to increase octane and cut down carbon monoxide and other smog-causing emissions. The most common blend of ethanol is E10 (10% ethanol, 90% gasoline). Some vehicles, called flexible fuel vehicles, are designed to run on E85 (a gasoline-ethanol blend containing 51%-83% ethanol, depending on geography and season), an alternative fuel with much higher ethanol content than regular gasoline. Roughly 97% of gasoline in the United States contains some ethanol. Most ethanol is made from plant starches and sugars, but cellulose and hemicellulose—the non-edible fibrous material that constitutes the bulk of plant matter—is also available as a biomass that can be deconstructed to component simple sugars for subsequent conversion into a biofuel.

Producing advanced biofuels (e.g., cellulosic ethanol and renewable hydrocarbon fuels) typically involves a multistep process. The common method for converting biomass into ethanol is called fermentation. During fermentation, microorganisms (e.g., bacteria and yeast) metabolize plant sugars and produce ethanol. First, the tough rigid structure of the plant cell wall—which includes the biological molecules cellulose, hemicellulose, and lignin bound tightly together—must be broken down. This can be generally accomplished in one of two ways: high temperature deconstruction or low temperature deconstruction.

High-Temperature Deconstruction

High-temperature deconstruction makes use of extreme heat and pressure to break down solid biomass into liquid or gaseous intermediates. There are three primary routes used in this pathway: pyrolysis, gasification, and hydrothermal liquefaction.

During pyrolysis, biomass is heated rapidly at high temperatures (500° C.-700° C.) in an oxygen-free environment. The heat generally breaks down biomass into pyrolysis vapor, gas, and char. Once the char is removed, the vapors are cooled and condensed into a liquid “bio-crude” oil.

Gasification generally follows a slightly similar process; however, biomass is exposed to a higher temperature range (>700° C.) with some oxygen present to produce synthesis gas (or syngas)—a mixture that consists mostly of carbon monoxide and hydrogen.

When working with wet feedstocks like algae, hydrothermal liquefaction is the preferred thermal process. This process uses water under moderate temperatures (200° C.-350° C.) and elevated pressures to convert biomass into liquid bio-crude oil.

Low-Temperature Deconstruction

Low-temperature deconstruction typically makes use of biological catalysts called enzymes or chemicals to breakdown feedstocks into intermediates. First, biomass undergoes a pretreatment step that opens up the physical structure of plant and algae cell walls, making sugar polymers like cellulose and hemicellulose more accessible. These polymers are then broken down enzymatically or chemically into simple sugar building blocks during a process known as hydrolysis.

Biological or Chemical Processing

Following deconstruction, intermediates such as crude bio-oils, syngas, sugars, and other chemical building blocks must be upgraded to produce a finished product. This step can involve either biological or chemical processing.

Microorganisms, such as bacteria, yeast, and cyanobacteria, can ferment sugar or gaseous intermediates into fuel blendstocks and chemicals. Alternatively, sugars and other intermediate streams, such as bio-oil and syngas, may be processed using a catalyst to remove any unwanted or reactive compounds in order to improve storage and handling properties. Nonetheless the microorganisms available to process the biomass into a biofuel must tolerate the harsh conditions required by the existing protocols. As the global demand for renewable energy solutions grows, the field of biofuels research has continued to seek solutions to meet these demands. The platform of the disclosure enables rapid and effective biofuel-relevant strain engineering by supporting a multiplex design and build of multiple target libraries—which enable the design of genetic variant libraries with a multitude of parallel insertions, swaps, and/or deletions, across multiple discrete and intertwined molecular pathways. These diverse cell populations engineered with the methods of the disclosure can yield individual improved strain variants with can be commercially suitable for the large scale production of biofuels.

The finished products from upgrading may be fuels or bioproducts ready to sell into the commercial market or stabilized intermediates suitable for finishing in a petroleum refinery or chemical manufacturing plant.

Biofuels—Biodiesel

Biodiesel is a liquid fuel produced from renewable sources, such as new and used vegetable oils and animal fats and is a cleaner-burning replacement for petroleum-based diesel fuel. Biodiesel is nontoxic and biodegradable and is produced by combining alcohol with vegetable oil, animal fat, or recycled cooking grease.

Like petroleum-derived diesel, biodiesel is used to fuel compression-ignition (diesel) engines. Biodiesel can be blended with petroleum diesel in any percentage, including B100 (pure biodiesel) and, the most common blend, B20 (a blend containing 20% biodiesel and 80% petroleum diesel).

Biofuels—Biogas

Biogas is gas produced by the process of anaerobic digestion of organic material by anaerobes, e.g., methane. Biogas can be produced either from biodegradable waste materials or by the use of energy crops fed into anaerobic digesters to supplement gas yields. The methods and platforms of the disclosure can be used in a screen to identify genetically engineered cells with improved abilities to perform, e.g., as the aforementioned anaerobic diesters. The solid byproduct, digestate, can be used as a biofuel or a fertilizer. A biogas that has CO₂ and other impurities removed from it is generally called biomethane.

Biogas can be recovered from mechanical biological treatment waste processing systems. Landfill gas, a less clean form of biogas, is produced in landfills through naturally occurring anaerobic digestion. If it escapes into the atmosphere, it may act as a greenhouse gas. Farmers can produce biogas from manure from their cattle by using anaerobic digesters.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Fully-Automated Singleplex RGN-Directed Editing Run

Singleplex automated genomic editing using MAD7 nuclease was successfully performed with an automated multi-module instrument as described in, e.g., U.S. Pat. No. 9,982,279; and U.S. Ser. No. 16/024,831 filed 30 Jun. 2018; Ser. No. 16/024,816 filed 30 Jun. 2018; Ser. No. 16/147,353 filed 28 Sep.2018; Ser. No. 16/147,865 filed 30 Sep. 2018; and Ser. No. 16/147,871 filed 30 Jun. 2018.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁻⁰³ fraction of cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example II: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in an isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

### Example III: Editing Cassette Preparation

5 nM oligonucleotides synthesized on a chip were amplified using Q5 polymerase in 50 μL volumes. The PCR conditions were 95° C. for 1 minute; 8 rounds of 95° C. for 30 seconds/60° C. for 30 seconds/72° C. for 2.5 minutes; with a final hold at 72° C. for 5 minutes. Following amplification, the PCR products were subjected to SPRI cleanup, where 30 μL SPRI mix was added to the 50 μL PCR reactions and incubated for 2 minutes. The tubes were subjected to a magnetic field for 2 minutes, the liquid was removed, and the beads were washed 2× with 80% ethanol, allowing 1 minute between washes. After the final wash, the beads were allowed to dry for 2 minutes, 50 μL 0.5× TE pH 8.0 was added to the tubes, and the beads were vortexed to mix. The slurry was incubated at room temperature for 2 minutes, then subjected to the magnetic field for 2 minutes. The eluate was removed and the DNA quantified.

Following quantification, a second amplification procedure was carried out using a dilution of the eluate from the SPRI cleanup. PCR was performed under the following conditions: 95° C. for 1 minute; 18 rounds of 95° C. for 30 seconds/72° C. for 2.5 minutes; with a final hold at 72° C. for 5 minutes. Amplicons were checked on a 2% agarose gel and pools with the cleanest output(s) were identified. Amplification products appearing to have heterodimers or chimeras were not used.

### Example IV: Backbone Preparation

Purified backbone vector was linearized by restriction enzyme digest with Stul. Up to 20 μg of purified backbone vector was in a 100 μL total volume in Stul-supplied buffer. Digestion was carried out at 30° C. for 16 hrs. Linear backbone was dialyzed to remove salt on 0.025 μm MCE membrane for ˜60 min on nuclease-free water. Linear backbone concentration was measured using dye/fluorometer-based quantification.

### Example V: Screening of Edited Libraries for Inhibitor Tolerance or Biofuel Production in E. coli

A selection was executed using several libraries of edited cells including a genome-wide knock out library and several different genome-wide promoter libraries at varying strengths (all libraries were built on E. coli strain (MG1655+Enginel)). The following set of compounds were tested at the listed concentration in triplicate, and a control flask with no compound added was also tested in parallel with the others in triplicate. These compounds were chosen because they're known biomass hydrolysate inhibitory compounds. The selection was carried out in minimal media for ˜48 hrs. Barcode amplicon data was measured and analyzed from the initial population of edited cells and from the end population following the selection.

| Compound | Concentration |
|---|---|
| HMF (5-hydroxymethyl furfural) | 1 g/L |
| Vanillin | 1 g/L |
| Syringic Acid | 1 g/L |
| Furfural | 1 g/L |
| Control population | No compound added |

A tolerance of toxic compounds formed or released during hydrolysis of a lignocellulosic biomass feed is measured based on cellular growth by the edited cells. Alternatively improved biofuel generation in an edited cell population is determined using a RapidFire high-throughput mass spectrometry system (Agilent) coupled to a 6470 Triple Quad mass spectrometer (Agilent). Suitable mass spectrometry conditions can be used for each biofuel.

### Example VI: Screening of Edited Libraries for Inhibitor Tolerance or Biofuel Production in Fermenting Microorganisms

The afternoon before transformation was to occur, 10 mL of YPAD was added to S. cerevisiae cells, and the culture was shaken at 250 rpm at 30° C. overnight. The next day, approximately 2 mL of the overnight culture was added to 100 mL of fresh YPAD in a 250-mL baffled flask and grown until the OD600 reading reached 0.3 +/−0.05. The culture was then placed in a 30° C. incubator shaking at 250 rpm and allowed to grow for 4-5 hours, with the OD checked every hour. When the culture reached ˜1.5 OD600, two 50 mL aliquots of the culture were poured into two 50-mL conical vials and centrifuged at 4300 rpm for 2 minutes at room temperature. The supernatant was removed from the 50 mL conical tubes, avoiding disturbing the cell pellet. 25 mL of lithium acetate/DTT solution was added to each conical tube and the pellet was gently resuspended using an inoculating loop, needle, or long toothpick.

Following resuspension, both cell suspensions were transferred to a 250-mL flask and placed in the shaker to shake at 30° C. and 200 rpm for 30 minutes. After incubation was complete, the suspension was transferred to one 50-mL conical tube and centrifuged at 4300 RPM for 3 minutes. The supernatant was then discarded. From this point on, cold liquids were used and kept on ice until electroporation was complete. 50 mL of 1 M sorbitol was added to the cells and the pellet was resuspended. The cells were centrifuged at 4300 rpm for 3 minutes at 4° C., and the supernatant was discarded. The centrifugation and resuspension steps were repeated for a total of three washes. 50 μL of 1 M sorbitol was then added to one pellet, the cells were resuspended, then this aliquot of cells was transferred to the other tube and the second pellet was resuspended. The approximate volume of the cell suspension was measured, then brought to a 1 mL volume with cold 1 M sorbitol. The cell/sorbitol mixture and transferred into a 2-mm cuvette. Impedance measurement of the cells was measured in the cuvette. At this point the KW must be ≥20. If this is not the case the cells should be washed in cold sorbitol two to three additional times.

Transformation was then performed using 500 ng of linear backbone along with 50 ng editing cassettes with the competent S. cerevisiae cells. 2 mm electroporation cuvettes were placed on ice and the plasmid/cassette mix was added to each corresponding cuvette. 100 μL of electrocompetent cells were added to each cuvette and the linear backbone and cassettes. Each sample was electroporated using the following conditions on a NEPAGENE electroporator: Poring pulse: 1800V, 5.0 second pulse length, 50.0 msec pulse interval, 1 pulse; Transfer pulse: 100 V, 50.0 msec pulse length, 50.0 msec pulse interval, with 3 pulses. Once the transformation process is complete, 900 μL of room temperature YPAD Sorbitol media was added to each cuvette. The cells were then transferred and suspended in a 15 mL tube and incubated shaking at 250 RPM at 30° C. for 3 hours. 9 mL of YPAD and 10 μL of Hygromycin B 1000× stock was added to the 15 mL tube.

Enhanced fermentation of the ethanol naturally produced by fermentation in Saccharomyces cerevisiae is demonstrated using the successful transfer and integration of the heterologous pathway for cellulase production into two model organisms for genome engineering. Specifically, the heterologous pathways for cellulase genes production are engineered into E. coli and S. cerevisiae using the automated methodologies described in more detail herein. This demonstrates Onyx's ability to rapidly, easily, and cost-effectively improve natural production of ethanol in both E. coli and S. cerevisiae.

By using a heterologous pathway for Ethanol production in S. cerevisiae, the resulting S. cerevisiae strain is capable of both biomass saccharification and ethanol fermentation, rather than requiring an upstream saccharification process for delivery of the fermentable sugars. Preferably, the heterologous pathways are engineered in the microbes that have also been engineered to have other desirable properties for biofuel generation.

Once the libraries are engineered for the production pathways, assays to detect enhanced production of ethanol will be used to identify cells that have the intended edits for enhanced production. These edits can be tracked within the positive cells using various assay and screening methods known to those in the art, e.g., mass spectrometry.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for generating engineered cells for the production of a biofuel comprising:
providing a population of cells;
generating the population of cells using an instrument for multiplexed nuclease-directed genome editing using introduced nucleic acids and a nucleic acid-directed nuclease to create cells, wherein the introduced nucleic acids comprise nucleic acids derived from a target library of genes;
incubating the processed cells to facilitate nucleic acid editing in the cell; and
selecting for edited cells displaying an improved production of the biofuel.

2. The method of claim 1, wherein the selecting selects for a cell that has improved resistance to an inhibitor generated during saccharification of biomass or in the production of the biofuel.

3. The method of claim 2, wherein the inhibitor is acetic acid, furfural, formic acid, hydroxymethyl furfural, vanillin, syringic acid, or levulinic acid.

4. The method of claim 1, wherein the selecting selects for a cell that has improved thermo-tolerance for the production of the biofuel.

5. The method of claim 1, wherein the selecting selects for a cell that has tolerance to a toxicity of the biofuel itself.

6. The method of claim 1, wherein the selecting selects for a cell that has a tolerance to a fermentation of a lignocellulosic substrate.

7. The method of claim 1, wherein the nuclease is an RNA-directed nuclease.

8. The method of claim 7, wherein the RNA-directed nuclease is selected from MAD7, Cas 9, Cas12/Cpf1.

9. The method of claim 1, wherein the library of target genes is a targeted library for generation of gene knock-outs in the population of cells.

10. The method of claim 1, wherein the library of target genes is a targeted library for generation of gene knock-ins in the population of cells.

11. The method of claim 9, wherein at least one gene in the targeted library for generation of gene knock-ins improves a secretion of the biofuel from the edited cells displaying the improved production of the biofuel.

12. The method of claim 9, wherein at least one gene in the targeted library for generation of gene knock-ins improves a tolerance to biomass inhibitors from the edited cells displaying the improved production of the biofuel.

13. The method of claim 1, wherein the cells are yeast cells.

14. A cell library produced using the method of claim 1.

15. A biofuel producing cell, wherein the cell is engineered via CRISPR mediated gene editing to introduce a non-endogenous biofuel pathway polynucleotide for translation into a functional polypeptide or set of polypeptides within the biofuel producing cell.

16. The biofuel producing cell of claim 15, wherein the biofuel producing cell lacks an endogenous pathway to produce the biofuel.

17. The biofuel producing cell of claim 15, wherein the cell is a microbial cell.

18. The biofuel producing cell of claim 16, wherein the cell is a bacterial cell.

19. The biofuel producing cell of claim 16, wherein the cell is a fungal cell.

20. The biofuel producing cell of claim 15, wherein the cell is a plant cell.

21. The biofuel producing cell of claim 15, wherein the non-endogenous biofuel pathway polynucleotide encodes one or more genes in an alcohol synthesis pathway.

22. The biofuel producing cell of claim 15, wherein the non-endogenous biofuel pathway polynucleotide encodes one or more genes in a diesel synthesis pathway.

23. The biofuel producing cell of claim 15, wherein the non-endogenous biofuel pathway polynucleotide encodes one or more genes in a biogas synthesis pathway.

24. The biofuel producing cell of claim 15, wherein the CRISPR mediated gene editing is multiplex gene editing.

25. The biofuel producing cell of claim 24, wherein two or more, three or more, or four or more non-endogenous biofuel pathway polynucleotides are engineered into the cell by the multiplex gene editing.

26. The biofuel producing cell of claim 15, wherein the biofuel producing cell has a high secretory phenotype for the biofuel as compared to a wild type cell.

27. The biofuel producing cell of claim 15, wherein the biofuel producing cell has a high fermentative capacity for the biofuel as compared to a wild type cell.

28. The biofuel producing cell of claim 15, wherein the biofuel producing cell has a resistance to bio-specific stressors for the biofuel as compared to a wild type cell.

29. The biofuel producing cell of claim 15, wherein the biofuel producing cell has a tolerance to products formed during pre-treatment and fermentation of lignocellulosic substrates should be used.

30. The biofuel producing cell of claim 15, wherein the non-endogenous biofuel pathway polypeptide is part of a pathway that processes a lignocellulosic biomass into the biofuel.
